SEQUENCE LISTING (42 sequences)

SEQ ID NO: 1 - DNA, 987 bp, Homo sapiens
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaa

SEQ ID NO: 2 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - DNA, 762 bp, Homo sapiens
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 4 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - DNA, 1008 bp, Mus musculus
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctaa

SEQ ID NO: 6 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 7 - DNA, 648 bp, Mus musculus
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaa

SEQ ID NO: 8 - protein, 215 aa, Mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 9 - DNA, 1602 bp, Artificial
  Fusion protein Construct ID 1481273
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacccctagcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttccagcggacccgctcccacctcagccacggtcatctgccgcaaaaatgccagcattagcgtgcgggcccaggaccgctactatagctcatcttggagcgaatgggcatctgtgccctgctcctaa

SEQ ID NO: 10 - protein, 533 aa, Artificial
  Fusion protein Construct ID 1481273
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTPSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGPAPTSATVICRKNASISVRAQDRYYSSSWSEWASVPCS

SEQ ID NO: 11 - DNA, 1602 bp, Artificial
  Fusion protein Construct ID 1481272
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctggctccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttccccccagacccctggccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcctaa

SEQ ID NO: 12 - protein, 533 aa, Artificial
  Fusion protein Construct ID 1481272
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASPQTPGPDTWSTPHSYFSLTFCVQVQGKSKREKKDRVFTDKTSATVICRKNASISVRAQDRYYSSSWSEWASVPCS

SEQ ID NO: 13 - DNA, 1623 bp, Artificial
  Fusion protein Construct ID 1480533
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagatcccccgtcagcggagacaatgccttccccgctcccacaggcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 14 - protein, 540 aa, Artificial
  Fusion protein Construct ID 1480533
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRSPVSGDNAFPAPTGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 15 - DNA, 1626 bp, Artificial
  Fusion protein Construct ID 1480534
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagatcccagcctgtgcctaccagggacctggaggtccccctcaccggaagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 16 - protein, 541 aa, Artificial
  Fusion protein Construct ID 1480534
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRSQPVPTRDLEVPLTGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 17 - DNA, 1629 bp, Artificial
  Fusion protein Construct ID 1480535
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagatccggcacaccccctcagacaggcctggagaagcccacaggcacaggcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 18 - protein, 542 aa, Artificial
  Fusion protein Construct ID 1480535
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRSGTPPQTGLEKPTGTGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 19 - DNA, 1623 bp, Artificial
  Fusion protein Construct ID 1480536
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagcgatgtgacaggcaataccggaaacgctacctataccattaccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 20 - protein, 540 aa, Artificial
  Fusion protein Construct ID 1480536
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSDVTGNTGNATYTITRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - DNA, 1623 bp, Artificial
  Fusion protein Construct ID 1480537
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggctcccccaaggacggacccgaaatccctcccacaggcggaaccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 22 - protein, 540 aa, Artificial
  Fusion protein Construct ID 1480537
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSPKDGPEIPPTGGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - DNA, 1629 bp, Artificial
  Fusion protein Construct ID 1480538
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggcaggaacgctcccggaagccctcccacaggcaattacaaactggaacccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 24 - protein, 542 aa, Artificial
  Fusion protein Construct ID 1480538
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGRNAPGSPPTGNYKLEPRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - DNA, 1632 bp, Artificial
  Fusion protein Construct ID 1480539
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcccagaaaggctccgtgggattcacagaccctgaggtccaccaaagcacaaacctcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 26 - protein, 543 aa, Artificial
  Fusion protein Construct ID 1480539
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSQKGSVGFTDPEVHQSTNLRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - DNA, 1623 bp, Artificial
  Fusion protein Construct ID 1480540
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggcaatgtgcctgagctgcccgataccacagagcatagcaggaccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 28 - protein, 540 aa, Artificial
  Fusion protein Construct ID 1480540
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGNVPELPDTTEHSRTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 29 - DNA, 1635 bp, Artificial
  Fusion protein Construct ID 1480541
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggcaggagccatcccgtccagccttaccctggcgctttcgtcaaggaacccattcccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 30 - protein, 544 aa, Artificial
  Fusion protein Construct ID 1480541
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGRSHPVQPYPGAFVKEPIPRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - DNA, 1623 bp, Artificial
  Fusion protein Construct ID 1480542
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcccccgaaagaaaagagaggatctccgagcaaacctatcagctgtccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 32 - protein, 540 aa, Artificial
  Fusion protein Construct ID 1480542
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSPERKERISEQTYQLSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 33 - DNA, 1623 bp, Artificial
  Fusion protein Construct ID 1480543
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggtggcggtggctcgggcggtggtgggtcgggtggcggcggatctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 34 - protein, 540 aa, Artificial
  Fusion protein Construct ID 1480543
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - DNA, 1599 bp, Artificial
  Fusion protein Construct ID 1480544
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggtggcggtggcggcggatctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 36 - protein, 532 aa, Artificial
  Fusion protein Construct ID 1480544
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 37 - DNA, 1653 bp, Artificial
  Fusion protein Construct ID 1480545
atgtgtccagcgcgcagcctcctccttgtggctaccctggtcctcctggaccacctcagtttggccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttccagatccgacgtgaactccagaacaggccctagcggagccacaccccctagcggcaacccctacacaatcacaggcatatgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcctaa

SEQ ID NO: 38 - protein, 550 aa, Artificial
  Fusion protein Construct ID 1480545
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASRSDVNSRTGPSGATPPSGNPYTITGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 39 - DNA, 1641 bp, Artificial
  Fusion protein Construct ID 1480546
atgtgtccagcgcgcagcctcctccttgtggctaccctggtcctcctggaccacctcagtttggccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcccccgctcccacaccctccaacggaagccctaaggatggccctgagattccccctaccggaggcatatgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcctaa

SEQ ID NO: 40 - protein, 546 aa, Artificial
  Fusion protein Construct ID 1480546
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASPAPTPSNGSPKDGPEIPPTGGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 3 aa, Artificial
  Synthetic linker
TPS

SEQ ID NO: 42 - protein, 5 aa, Artificial
  Synthetic linker
SGPAP

(19) United States
(12) Patent Application Publication
(10) Pub. No.: US 2016/0311879 A1
(43) Pub. Date: Oct. 27, 2016

(54) SINGLE CHAIN IL-12 NUCLEIC ACIDS, POLYPEPTIDES, AND USES THEREOF

(71) Applicant: INTREXON CORPORATION, Blacksburg, VA (US)

(72) Inventors: Joan Mazzarelli SOPCZYNSKI, Oreland, PA (US); Chi ZHANG, Omaha, NE (US)

(73) Assignee: Intrexon Corporation, Blacksburg, VA (US)

(21) Appl. No.: 15/105,435

(22) PCT Filed: Dec. 17, 2014
(86) PCT No.: PCT/US2014/070695

§ 371 Date: Jun. 16, 2016

Related U.S. Application Data
(60) Provisional application No. 61/917,495, filed on Dec. 18, 2013.

(51) Int. Cl. C07K 14/54

(52) U.S. Cl. CPC C07K 14/5434; A61K 38/00

(57) ABSTRACT

The present invention relates to novel single chain interleukin-12 polypeptides. The invention also relates to isolated nucleic acids encoding the single chain interleukin-12 polypeptides, to vectors and cells comprising them, and to their uses, in particular in methods of using single chain IL-12 polypeptides, polynucleotides, vectors and cells of the invention for enhancing immune system function, for example as vaccine adjuvants and in the treatment of infections and cancer.

### REFERENCE TO SEQUENCE LISTING

The content of the electronically submitted sequence listing (Name: SequenceListing.txt; Size: 127,325 bytes; Date of Creation: Dec. 16, 2013) filed with this application is incorporated herein by reference in its entirety.

### FIELD OF THE INVENTION

The present invention provides novel nucleic acids encoding single-chain interleukin-12 fusion proteins, vectors comprising them, polypeptides encoded by them, and for their use in therapeutic applications.

### BACKGROUND OF THE INVENTION

Interleukin-12 (IL-12) is an inflammatory cytokine that is produced in response to infection by a variety of cells of the immune system, including phagocytic cells, B cells and activated dendritic cells (Colombo and Trinchieri (2002), Cytokine & Growth Factor Reviews, 13: 155-168). IL-12 plays an essential role in mediating the interaction of the innate and adaptive arms of the immune system, acting on T-cells and natural killer (NK) cells, enhancing the proliferation and activity of cytotoxic lymphocytes and the production of other inflammatory cytokines, especially interferon-gamma (IFN-gamma).

IL-12 has been tested in human clinical trials as an immunotherapeutic agent for the treatment of a wide variety of cancers (Atkins et al. (1997), Clin. Cancer Res., 3: 409-17; Gollob et al. (2000), Clin. Cancer Res., 6: 1678-92; Hurteau et al. (2001), Gynecol. Oncol., 82: 7-10; and Youssoufian, et al. (2013) Surgical Oncology Clinics of North America, 22(4): 885-901), including renal, colon, and ovarian cancer, melanoma and T-cell lymphoma, and as an adjuvant for cancer vaccines (Lee et al. (2001), J. Clin. Oncol. 19: 3836-47). However, IL-12 is toxic when administered systemically as a recombinant protein. Trinchieri, Adv. Immunol. 1998; 70:83-243. In order to maximize the anti-tumoral effect of IL-12 while minimizing its systemic toxicity, IL-12 gene therapy approaches have been proposed to allow production of the cytokine at the tumor site, thereby achieving high local levels of IL-12 with low serum concentration. Qian et al., Cell Research (2006) 16: 182-188; US Patent Publication 20130195800.

IL-12 is a heterodimeric molecule composed of an alpha chain (the p35 subunit) and a beta chain (the p40 subunit) covalently linked by a disulfide bridge to form the biologically active 70 kD a dimer. Simultaneous expression of the two subunits is necessary for the production of the biologically active heterodimer. Recombinant IL-12 expression has been achieved using bicistronic vectors containing the p40 and p35 subunits separated by an IRES (internal ribosome entry site) sequence to allow independent expression of both subunits from a single vector. However, the use of IRES sequences can impair protein expression. Mizuguchi et al. Mol Ther (2000); 1: 376-382. Moreover, unequal expression of the p40 and p35 subunits can lead to the formation of homodimeric proteins (e.g. p40-p40) which can have inhibitory effects on IL-12 signaling. Gillessen et al. Eur. J. Immunol. 1995 Jan;25(1):200-6.

As an alternative to bicistronic expression of the IL-12 subunits, functional single chain IL-12 fusion proteins have been produced by joining the p40 and p35 subunits with (Gly4Ser)3 or Gly6Ser linkers. Lieschke et al., (1997), Nature Biotechnology 15, 35-40; Lode et al., (1998), PNAS 95, 2475 -2480. However, longer linker sequences may interfere with the ability to construct viral vectors for gene therapy, and may increase the likelihood of inducing immunogenic responses (e.g., by generating anti-single chain IL-12 antibodies).

Therefore, there remains a need in the art for improved single chain IL-12 fusion proteins and nucleic acids encoding such fusion proteins for use in enhancing immune system function, for example as vaccine adjuvants and in the treatment of infections and cancer.

### BRIEF SUMMARY OF THE INVENTION

The present invention relates to novel single chain IL-12 (scIL-12) polypeptides wherein the length of linker sequences, if any, is minimized by inserting IL-12 p35 polypeptide sequences within an IL-12 p40 polypeptide sequence while retaining at least one IL-12 biological activity.

The present invention relates to scIL-12 polypeptides comprising, from N- to C-terminus:

(i) a first IL-12 p40 domain (p40N),

(ii) an optional first peptide linker,

(iii) an IL-12 p35 domain,

(iv) a optional second peptide linker, and

(v) a second IL-12 p40 domain (p40C).

In preferred embodiments, scIL-12 polypeptides of the invention retain at least one biological activity of IL-12.

The invention further relates to scIL-12 polynucleotides encoding scIL-12 polypeptides as described herein, and to vectors comprising said scIL-12 polynucleotides.

The invention also relates to variant scIL-12 polypeptides having 80%, 85%, 90%, or 95% identity to a scIL-12 polypeptide disclosed herein.

The invention also relates to a cell or a non-human organism transformed or transfected with a scIL-12 polynucleotide or vector as described herein.

The invention also relates to a pharmaceutical or diagnostic composition comprising as an active agent a scIL-12 polypeptide, polynucleotide, vector, or cell as described herein.

The invention also relates to methods of using scIL-12 polypeptides, polynucleotides, vectors and cells of the invention for enhancing immune system function, for example as vaccine adjuvants and in the treatment of infections and cancer.

### DESCRIPTION OF THE FIGURES

FIG. 1. Schematic diagrams showing the p40-p35 single chain configuration (FIG. 1A), the p35-p40 single chain configuration (FIG. 1B), and a p40N-p35-p40C insert configuration (FIG. 1 C). The construction and characterization of these designs are discussed in detail in the Examples.

FIG. 2. Expression levels of human scIL-12 designs as determined by p70 ELISA (see Example 2).

FIG. 3. scIL-12-stimulated IFN-gamma production, as measured by ELISA (see Example 3).

FIG. 4. In vitro dose-dependent expression of interferon-gamma (i.e., “IFN-gamma,” “IFN-g” or “IFN-y”) by NK92 cells exposed to recombinant human or mouse IL-12 p40/p35 heterodimeric polypeptides (see Example 4).

FIG. 5. In vitro dose-dependent expression of interferon-gamma by NK92 cells exposed to heterodimeric IL-12 p40/p35 polypeptides and single chain IL-12 polypeptides (including p40N-p35-p40C single chain IL-12 (SEQ ID NO:10)); (see Example 5).

### DETAILED DESCRIPTION OF THE INVENTION

The present invention advantageously provides isolated polynucleotides encoding single chain IL-12 (scIL-12) polypeptides. The polynucleotides and polypeptides of the present invention are useful in methods of enhancing the immune response of a host, for example as vaccine adjuvants, and in the treatment of proliferative disorders such as cancer, infectious diseases, and immune system disorders.

The various aspects of the invention will be set forth in greater detail in the following sections, directed to the nucleic acids, polypeptides, vectors, compositions, antibodies and methods of use of the invention. This organization into various sections is intended to facilitate understanding of the invention, and is in no way intended to be limiting thereof.

### Definitions

The following defined terms are used throughout the present specification, and should be helpful in understanding the scope and practice of the present invention.

In a specific embodiment, the term “about” or “approximately” means within 20%, preferably within 10%, more preferably within 5%, and even more preferably within 1% of a given value or range.

The term “substantially free” means that a composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

The term “isolated” for the purposes of the present invention designates a biological material (nucleic acid or protein) that has been removed from its original environment (the environment in which it is naturally present). F or example, a polynucleotide present in the natural state in a plant or an animal is not isolated, however the same polynucleotide separated from the adjacent nucleic acids in which it is naturally present, is considered “isolated”. The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

As used herein, the term “substantially pure” describes a polypeptide or other material which has been separated from its native contaminants. Typically, a monomeric polypeptide is substantially pure when at least about 60 to 75% of a sample exhibits a single polypeptide backbone. Minor variants or chemical modifications typically share the same polypeptide sequence. Usually a substantially pure polypeptide will comprise over about 85 to 90% of a polypeptide sample, and preferably will be over about 99% pure. Normally, purity is measured on a polyacrylamide gel, with homogeneity determined by staining Alternatively, for certain purposes high resolution will be necessary and HPLC or a similar means for purification will be used. For most purposes, a simple chromatography column or polyacrylamide gel will be used to determine purity.

The term “substantially free of naturally-associated host cell components” describes a polypeptide or other material which is separated from the native contaminants which accompany it in its natural host cell state. Thus, a polypeptide which is chemically synthesized or synthesized in a cellular system different from the host cell from which it naturally originates will be free from its naturally-associated host cell components.

The terms “nucleic acid” or “polynucleotide” are used interchangeably herein to refer to a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes but is not limited to cDNA, genomic DNA, plasmid DNA, synthetic DNA, and semi-synthetic DNA. DNA may be linear, circular, or supercoiled.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes, without limitation, double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” will be understood to mean a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6-1500 consecutive nucleotides of a nucleic acid according to the invention.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to an assembly of nucleotides that encode an RNA transcript or a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Heterologous” DNA refers to DNA not naturally located in the cell, or in a chromosomal site of the cell. Preferably, the heterologous DNA includes a gene foreign to the cell.

The term “genome” includes chromosomal as well as mitochondrial, chloroplast and viral DNA or RNA.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989 infra). Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization.

Stringency conditions can be adjusted to screen for moderately similar fragments, such as homologous sequences from distantly related organisms, to highly similar fragments, such as genes that duplicate functional enzymes from closely related organisms. F or preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC.

Hybridization requires that the two nucleic acids contain complementary sequences, although depending on t he stringency of the hybridization, mismatches between bases are possible. The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as disclosed or used herein as well as those substantially similar nucleic acid sequences.

In a specific embodiment of the invention, polynucleotides are detected by employing hybridization conditions comprising a hybridization step at Tm of 55° C., and utilizing conditions as set forth above. In certain embodiments, the Tm is 60° C., 63° C. or 65° C.

Post-hybridization washes also determine stringency conditions. In certain embodiments the hybridization conditions use a series of washes starting with 6×SSC, 0.5% SDS at room temperature for 15 minutes (min), then repeated with 2×SSC, 0.5% SDS at 45° C. for 30 minutes, and then repeated twice with 0.2×SSC, 0.5% SDS at 50° C. for 30 minutes. A more stringent set of conditions uses higher temperatures in which the washes are identical to those above except for the temperature of the final two 30 min washes in 0.2×SSC, 0.5% SDS is increased to 60° C. A highly stringent set of conditions uses two final washes in 0.1×SSC, 0.1% SDS at 65° C.

The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8).

Selectivity of hybridization exists when hybridization occurs which is more selective than total lack of specificity. Typically, selective hybridization will occur when there is at least about 55% homology over a stretch of at least about 14/25 nucleotides, preferably at least about 65%, more preferably at least about 75%, and most preferably at least about 90%. See, Kanehisa, M. (1984), Nucleic Acids Res. 12:203-213, which is incorporated herein by reference. Stringent hybridization conditions will typically include salt concentrations of less than about 1 M , more usually less than about 500 mM and preferably less than about 200 mM. Temperature conditions will typically be greater than 20 degrees Celsius, more usually greater than about 30 degrees Celsius and preferably in excess of about 37 degrees Celsius. As other factors may significantly affect the stringency of hybridization, including, among others, base composition and size of the complementary strands, presence of organic solvents and extent of base mismatching, the combination of parameters is more important than the absolute measure of any one.

In a specific embodiment of the invention, polynucleotides of the invention are detected by employing hybridization conditions comprising a hybridization step in less than 500 mM salt and at least 37 degrees Celsius, and a washing step in 2×SSPE at least 63 degrees Celsius. In certain embodiment, the hybridization conditions comprise less than 200 mM salt and at least 37 degrees Celsius for the hybridization step. In another embodiment, the hybridization conditions comprise 2×SSPE and 63 degrees Celsius for both the hybridization and washing steps.

In one embodiment, the length for a hybridizable nucleic acid is at least about 10 nucleotides. Preferable a minimum length for a hybridizable nucleic acid is at least about 15 nucleotides; more preferably at least about 20 nucleotides; and even more preferably the length is at least 30 nucleotides. Furthermore, the skilled artisan will recognize that the temperature and wash solution salt concentration may be adjusted as necessary according to factors such as length of the probe.

The term “probe” refers to a single-stranded nucleic acid molecule that can base pair with a complementary single stranded target nucleic acid to form a double-stranded molecule.

As used herein, the term “oligonucleotide” refers to a nucleic acid, generally of at least 18 nucleotides, that is hybridizable to a genomic DNA molecule, a cDNA molecule, a plasmid DNA or an mRNA molecule. Oligonucleotides can be labeled, e.g., with ³²P-nucleotides or nucleotides to which a label, such as biotin, has been covalently conjugated. A labeled oligonucleotide can be used as a probe to detect the presence of a nucleic acid. Oligonucleotides (one or both of which may be labeled) can be used as PCR primers, either for cloning full length or a fragment of a nucleic acid, or to detect the presence of a nucleic acid. An oligonucleotide can also be used to form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “primer” is an oligonucleotide that hybridizes to a target nucleic acid sequence to create a double stranded nucleic acid region that can serve as an initiation point for DNA synthesis under suitable conditions. Such primers may be used in a polymerase chain reaction.

“Polymerase chain reaction” is abbreviated PCR and means an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase. PCR provides a means to detect the presence of the target molecule and, under quantitative or semi-quantitative conditions, to determine the relative amount of that target molecule within the starting pool of nucleic acids.

“Reverse transcription-polymerase chain reaction” is abbreviated RT-PCR and means an in vitro method for enzymatically producing a target cDNA molecule or molecules from an RNA molecule or molecules, followed by enzymatic amplification of a specific nucleic acid sequence or sequences within the target cDNA molecule or molecules as described above. RT-PCR also provides a means to detect the presence of the target molecule and, under quantitative or semi-quantitative conditions, to determine the relative amount of that target molecule within the starting pool of nucleic acids.

A DNA “coding sequence” is a double-stranded DNA sequence that is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, without limitation, promoters, translation leader sequences, introns, polyadenylation recognition sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and means a length of nucleic acid sequence, either DNA, cDNA or RNA, that comprises a translation start signal or initiation codon, such as an ATG or AUG, and a termination codon and can be potentially translated into a polypeptide sequence.

The term “head-to-head” is used herein to describe the orientation of two polynucleotide sequences in relation to each other. Two polynucleotides are positioned in a head-to-head orientation when the 5′ end of the coding strand of one polynucleotide is adjacent to the 5′ end of the coding strand of the other polynucleotide, whereby the direction of transcription of each polynucleotide proceeds away from the 5′ end of the other polynucleotide. The term “head-to-head” may be abbreviated (5′)-to-(5′) and may also be indicated by the symbols (← →) or (3′←5′5′→3′).

The term “tail-to-tail” is used herein to describe the orientation of two polynucleotide sequences in relation to each other. Two polynucleotides are positioned in a tail-to-tail orientation when the 3′ end of the coding strand of one polynucleotide is adjacent to the 3′ end of the coding strand of the other polynucleotide, whereby the direction of transcription of each polynucleotide proceeds toward the other polynucleotide. The term “tail-to-tail” may be abbreviated (3′)-to-(3′) and may also be indicated by the symbols (→ ←) or (5′→3′3′←5′).

The term “head-to-tail” is used herein to describe the orientation of two polynucleotide sequences in relation to each other. Two polynucleotides are positioned in a head-to-tail orientation when the 5′ end of the coding strand of one polynucleotide is adjacent to the 3′ end of the coding strand of the other polynucleotide, whereby the direction of transcription of each polynucleotide proceeds in the same direction as that of the other polynucleotide. The term “head-to-tail” may be abbreviated (5 ‘)-to-(3’) and may also be indicated by the symbols (→ →) or (5′→3′5′→3′).

The term “downstream” refers to a nucleotide sequence that is located 3′ to reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to reference nucleotide sequence. In particular, upstream nucleotide sequences generally relate to sequences that are located on t he 5′ side of a coding sequence or starting point of transcription. For example, most promoters are located upstream of the start site of transcription.

The terms “restriction endonuclease” and “restriction enzyme” refer to an enzyme that binds and cuts within a specific nucleotide sequence within double stranded DNA.

“Homologous recombination” refers to the insertion of a foreign DNA sequence into another DNA molecule, e.g., insertion of a vector in a chromosome. Preferably, the vector targets a specific chromosomal site for homologous recombination. F or specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

Many methods known in the art may be used to propagate a polynucleotide according to the invention. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus, adenovirus and adeno-associated virus (AAV); insect viruses such as baculovirus; yeast vectors; bacteriophage vectors (e.g., lambda); and plasmid and cosmid DNA vectors, to name but a few.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. A large number of vectors known in the art may be used to manipulate nucleic acids, incorporate response elements and promoters into genes, etc. Possible vectors include, for example but without limitation, plasmids or modified viruses including, for example bacteriophages such as lambda derivatives, or plasmids such as pBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include but are not limited to retrovirus, adeno-associated virus (AAV), pox, baculovirus, vaccinia, herpes simplex, Epstein-Barr, adenovirus, geminivirus, and caulimovirus vectors. Non-viral vectors include, without limitation, plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “cloning vector” is a “replicon”, which is a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”).

Vectors may be introduced into the desired host cells by methods known in the art, e.g., transfection, electroporation, microinjection, transduction, cell fusion, DEAE dextran, calcium phosphate precipitation, lipofection (lysosome fusion), particle bombardment, use of a gene gun, or a DNA vector transporter (see, e.g., Wu et al., 1992, J. Biol. Chem. 267:963-967; Wu and Wu, 1988, J. Biol. Chem. 263:14621-14624; and Hartmut et al., Canadian Patent Application No. 2,012,311, filed Mar. 15, 1990).

A polynucleotide according to the invention can also be introduced in vivo by lipofection. F or the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et al., 1987. PNAS 84:7413; Mackey, et al., 1988. Proc. Natl. Acad. Sci. U.S.A. 85:8027-8031; and Ulmer et al., 1993. Science 259:1745-1748). The use of cationic lipids may promote encapsulation of negatively charged nucleic acids, and also promote fusion with negatively charged cell membranes (Felgner and Ringold, 1989. Science 337:387-388). Particularly useful lipid compounds and compositions for transfer of nucleic acids are described in International Patent Publications WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce a vector in vivo as a naked DNA plasmid (see U.S. Pat. Nos. 5,693,622, 5,589,466 and 5,580,859). Receptor-mediated DNA delivery approaches can also be used (Curiel et al., 1992. Hum. Gene Ther. 3:147-154; and Wu and Wu, 1987. J. Biol. Chem. 262:4429-4432).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Transformation” refers to the transfer of a nucleic acid molecule into the genome of a host organism, resulting in genetically stable inheritance. Host organisms containing the transformed nucleic acid molecule are referred to as “transgenic” or “recombinant” or “transformed” organisms.

The term “genetic region” will refer to a region of a nucleic acid molecule or a nucleotide sequence that comprises a gene encoding a polypeptide.

In addition, the recombinant vector comprising a polynucleotide according to the invention may include one or more origins for replication in the cellular hosts in which their amplification or their expression is sought, markers or selectable markers.

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include, without limitation: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Selectable marker genes may also be considered reporter genes.

The term “reporter gene” means a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include, without limitation: luciferase (Luc), green fluorescent protein (GFP), chloramphenicol acetyltransferase (CAT), β-galactosidase (LacZ), β-glucuronidase (Gus), and the like.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. Promoters that cause a gene to be expressed in a specific cell type are commonly referred to as “cell-specific promoters” or “tissue-specific promoters”. Promoters that cause a gene to be expressed at a specific stage of development or cell differentiation are commonly referred to as “developmentally-specific promoters” or “cell differentiation-specific promoters”. Promoters that are induced and cause a gene to be expressed following exposure or treatment of the cell with an agent, biological molecule, chemical, ligand, light, or the like that induces the promoter are commonly referred to as “inducible promoters” or “regulatable promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “response element” means one or more cis-acting DNA elements which confer responsiveness on a promoter mediated through interaction with the DNA-binding domains of the first chimeric gene. This DNA element may be either palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon t he nature of the cell or organism into which the response element will be incorporated. The DNA binding domain of the first hybrid protein binds, in the presence or absence of a ligand, to the DNA sequence of a response element to initiate or suppress transcription of downstream gene(s) under the regulation of this response element.

The term “operably linked” refers to the association of nucleic acid sequences on a single nucleic acid fragment so that the function of one is affected by the other. F or example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from a nucleic acid or polynucleotide. Expression may also refer to translation of mRNA into a protein or polypeptide.

The terms “cassette”, “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

The terms “modulate” and “modulates” mean to induce, reduce or inhibit nucleic acid or gene expression, resulting in the respective induction, reduction or inhibition of protein or polypeptide production.

The plasmids or vectors according to the invention may further comprise at least one promoter suitable for driving expression of a gene in a host cell. The term “expression vector” means a vector, plasmid or vehicle designed to enable the expression of an inserted nucleic acid sequence following transformation into the host. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving these genes is suitable for the present invention including but not limited to: viral promoters, bacterial promoters, animal promoters, mammalian promoters, synthetic promoters, constitutive promoters, tissue specific promoter, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TP1, alkaline phosphatase promoters (useful for expression in Saccharomyces); AOX1 promoter (useful for expression in Pichia); b-lactamase, lac, ara, tet, trp, lP_{L}, lP_{R}, T7, tac, and trc promoters (useful for expression in Escherichia coli); light regulated-, seed specific-, pollen specific-, ovary specific-, pathogenesis or disease related-, cauliflower mosaic virus 35S, CMV 35S minimal, cassava vein mosaic virus (CsVMV), chlorophyll a/b binding protein, ribulose 1, 5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art include, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus IE1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell a-actin, and the like. In addition, these expression sequences may be modified by addition of enhancer or regulatory sequences and the like.

Enhancers that may be used in embodiments of the invention include but are not limited to: an SV40 enhancer, a cytomegalovirus (CMV) enhancer, an elongation factor 1 (EF1) enhancer, yeast enhancers, viral gene enhancers, and the like.

Termination control regions, i.e., terminator or polyadenylation sequences, may also be derived from various genes native to the preferred hosts. Optionally, a termination site may be unnecessary, however, it is most preferred if included. In certain embodiments of the invention, the termination control region may be comprise or be derived from a synthetic sequence, synthetic polyadenylation signal, an SV40 late polyadenylation signal, an SV40 polyadenylation signal, a bovine growth hormone (BGH) polyadenylation signal, viral terminator sequences, or the like.

The terms “3′ non-coding sequences” or “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Regulatory region” means a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include, without limitation, origins of replication, RNA splice sites, promoters, enhancers, transcriptional termination sequences, and signal sequences which direct the polypeptide into the secretory pathways of the target cell.

A regulatory region from a “heterologous source” is a regulatory region that is not naturally associated with the expressed nucleic acid. Included among the heterologous regulatory regions are, without limitation, regulatory regions from a different species, regulatory regions from a different gene, hybrid regulatory sequences, and regulatory sequences which do not occur in nature, but which are designed by one having ordinary skill in the art.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene. The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

A “polypeptide” is a polymeric compound comprised of covalently linked amino acid residues. Amino acids have the following general structure:

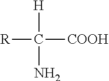

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

An “isolated polypeptide” or “isolated protein” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

A “fragment” of a polypeptide according to the invention will be understood to mean a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of at least 2-300 amino acids.

A “heterologous protein” refers to a protein not naturally produced in the cell.

A “mature protein” refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

The term “homology” refers to the percent of identity between two polynucleotide or two polypeptide moieties. The correspondence between the sequence from one moiety to another can be determined by techniques known to the art. F or example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667.). Such proteins (and their encoding genes) have sequence homology, as reflected by their high degree of sequence similarity. However, in common usage and in the instant application, the term “homologous,” when modified with an adverb such as “highly,” may refer to sequence similarity and not a common evolutionary origin.

Accordingly, the term “sequence similarity” in all its grammatical forms refers to the degree of identity or correspondence between nucleic acid or amino acid sequences of proteins that may or may not share a common evolutionary origin (see Rccck et al., 1987, Cell 50:667).

In a specific embodiment, two DNA sequences are “substantially homologous” or “substantially similar” when at least about 50% (preferably at least about 75%, and most preferably at least about 90 or 95%) of the nucleotides match over the defined length of the DNA sequences.

Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Sambrook et al., 1989, supra.

As used herein, “substantially similar” refers to nucleic acid fragments wherein changes in one or more nucleotide bases results in substitution of one or more amino acids, but do not affect the functional properties of the protein encoded by the DNA sequence. “Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotide bases that do not substantially affect the functional properties of the resulting transcript. I t is therefore understood that the invention encompasses more than the specific exemplary sequences. Each of the proposed modifications is well within the routine skill in the art, as is determination of retention of biological activity of the encoded products.

Moreover, the skilled artisan recognizes that substantially similar sequences encompassed by this invention are also defined by their ability to hybridize, under stringent conditions (0.1×SSC, 0.1% SDS, 65° C. and washed with 2×SSC, 0.1% SDS followed by 0.1×SSC, 0.1% SDS), with the sequences exemplified herein. Substantially similar nucleic acid fragments of the instant invention are those nucleic acid fragments whose DNA sequences are at least 70% identical to the DNA sequence of the nucleic acid fragments reported herein. Substantially similar nucleic acid fragments of the instant invention include those nucleic acid fragments whose DNA sequences are at least 80% identical to the DNA sequence of the nucleic acid fragments reported herein. In certain embodiments nucleic acid fragments are at least 90% identical, at least 95% identical, at least 97% identical, at least 98% identical, or at least 99% identical to the DNA sequence of the nucleic acid fragments reported herein. In certain embodiments, substantially similar nucleotide sequences of the invention can encode any polypeptide sequences described in the present application (e.g., scIL-12 polypeptides) despite any differences in nucleotide sequences present in comparison to specific polynucleotide sequences described herein.

Two amino acid sequences are “substantially homologous” or “substantially similar” when greater than about 40% of the amino acids are identical, or greater than 60% are similar (functionally identical). Preferably, the similar or homologous sequences are identified by alignment using, for example, the GCG (Genetics Computer Group, Program Manual for the GCG Package, Version 7, Madison, Wis.) pileup program.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A “substantial portion” of an amino acid or nucleotide sequence comprises enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method may be selected: KTUPLE 1, GAP PENALTY=3, WINDOW=5 and DIAGONALS SAVED=5.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available. Alternatively, or in addition to optimization to reflect codon bias, optimization can also include optimization of nucleotide sequence based on specific host cells wherein optimization is performed to maximize transcription rate or quantity, transcript half-life, and translation rate or quantity. Such optimization can be performed through empirical determinations based on specific host cell.

The term “gene switch” refers to the combination of a response element associated with a promoter, and a ligand-dependent transcription factor-based system which, in the presence of one or more ligands, modulates the expression of a gene with which the response element and promoter are operably associated. The term “a polynucleotide encoding a gene switch” refers to the combination of a response element associated with a promoter, and a polynucleotide encoding a ligand-dependent transcription factor-based system which, in the presence of one or more ligands, modulates the expression of a gene with which the response element and promoter are operably associated.

The terms “IL-12 activity” and “IL-12 biological activity” refer to any of the well-known bioactivities of IL-12, and include, without limitation, stimulating differentiation of naive T cells into Th1 cells, stimulating growth and function of T cells, stimulating production of interferon-gamma (IFN-gamma) and tumor necrosis factor-alpha (TNF-alpha) from T-cells and natural killer (NK) cells, stimulating reduction of IL-4 mediated suppression of IFN-gamma, stimulating enhancement of the cytotoxic activity of NK cells and CD8+ cytotoxic T lymphocytes, stimulating expression of IL-12R-betal and IL-12R-beta2, facilitating the presentation of tumor antigens through the upregulation of MHC I and II molecules, and stimulating anti-angiogenic activity. Exemplary assays for IL-12 activity include the Gamma Interferon Induction Assay (see Example 3, and U.S. Pat. No. 5,457,038). Additional assays are known in the art, such as, but not limited to, NK Cell Spontaneous Cytotoxicity Assays, ADCC Assays, Co-Mitogenic Effect Assays, and GM-CSF Induction Assays (e.g., as disclosed in Example 8 of U.S. Pat. No. 5,457,038, incorporated herein by reference).

In a preferred embodiment, scIL-12 polypeptides of the invention retain at least one IL-12 biological activity. In certain embodiments, scIL-12 polypeptides of the invention retain more than one IL-12 biological activity. In certain embodiments, scIL-12 polypeptides of the invention retain at least one, at least two, at least three, at least four, at least five or at least six of the above-referenced IL-12 biological activities. In certain embodiments, the IL-12 biological activity of scIL-12 polypeptides of the present invention is compared to (assayed against) the heterodimeric p35/p40 (wild-type) form of IL-12. In certain embodiments, scIL-12 polypeptides of the invention retain at least about 50%, at least about 75%, at least about 85%, at least about 90%, at least about 100%, at least 50%, at least 75%, at least 85%, at least 90%, at least 100%, or more of the biological activity of IL-12 compared to the heterodimeric p35/p40 (wild-type) form of IL-12.

As used herein, the terms “treating” or “treatment” of a disease refer to executing a protocol, which may include administering one or more drugs or in vitro engineered cells to a mammal (human or non-human), in an effort to alleviate signs or symptoms of the disease. Thus, “treating” or “treatment” should not necessarily be construed to require complete alleviation of signs or symptoms, does not require a cure, and specifically includes protocols that have only marginal effect on the subject.

As used herein, “immune cells” include dendritic cells, macrophages, neurophils, mast cells, eosinophils, basophils, natural killer cells and lymphocytes (e.g., B and T cells).

As used herein, the term “stem cells” includes embryonic stem cells, adult stem cells and induced pluripotent stem cells. Stem cells can be obtained from any appropriate source, including bone marrow, adipose tissue, and blood (including, but not limited to, umbilical cord blood and menstrual blood). Examples of stem cells include, but are not limited to, mesenchymal stem cells and hematopoietic stem cells.

As used herein, the terms “dendritic cells” and “DC” are interchangeably used. Likewise, the terms “Natural Killer Cells” and “NK cells” are interchangeably used. Polynucleotides Encoding Single Chain IL-12 Polypeptides

The present invention provides novel polynucleotides encoding a single chain interleukin-12 (scIL-12) polypeptide of the invention, including full length and mature scIL-12 polypeptides.

In accordance with specific embodiments of the present invention, nucleic acid sequences encoding novel scIL-12 polypeptides are provided. Specifically, the invention provides polynucleotides encoding a scIL-12 polypeptide comprising, from N- to C-terminus:

(i) a first IL-12 p40 domain (p40N),

(ii) an optional first peptide linker,

(iii) an IL-12 p35 domain,

(iv) an optional second peptide linker, and

(v) a second IL-12 p40 domain (p40C).

In certain embodiments, the first IL-12 p40 domain (also referred to herein as p40N) encoded by polynucleotides of the invention is an N-terminal fragment of an IL-12 p40 subunit. IL-12 p40 polynucleotides for use in the invention include the human IL-12 p40 nucleic acid sequence of SEQ ID NO: 1 and the murine IL-12 p40 nucleic acid sequence of SEQ ID NO: 5. Additional, non-limiting examples of polynucleotides encoding IL-12 p40 subunits are available in public sequence databases, including but not limited to Genbank Accession Nos. AF180563.1 (human), NM_002187.2 (human), NG_009618.1 (human), NM_001077413.1 (cat), AF091134.1 (dog), NM_008352.2 (mouse), NM_001159424.1 (mouse), and NM_008351.2 (mouse).

N-terminal fragments of IL-12 p40 encoded by polynucleotides of the invention and suitable as a first IL-12 p40 domain (p40N) include, but are not limited to, polypeptides comprising, or alternatively consisting of, amino acids 1 to 288, 1 to 289, 1 to 290, 1 to 291, 1 to 292, 1 to 293, 1 to 294, 1 to 295, 1 to 296, 1 to 297, and 1 to 298 of SEQ ID NO: 2. A preferred N-terminal fragment of IL-12 p40 encoded by polynucleotides of the invention and suitable as a first IL-12 p40 domain (p40N) comprises, or alternatively consists of, amino acids 1 to 293 of SEQ ID NO: 2.

N-terminal fragments of IL-12 p40 encoded by polynucleotides of the invention and suitable as a first IL-12 p40 domain (p40N) may lack a signal sequence. It is understood that the specific cleavage site of a signal peptide may vary by 1, 2, 3 or more residues. Accordingly, in additional embodiments the first IL-12 p40 domain (p40N) encoded by polynucleotides of the invention comprises, or alternatively consists of, a fragment of SEQ ID NO: 2 beginning with residue 18, 19, 20, 21, 22, 23, 24, 25, 26, 27 or 28 of SEQ ID NO: 2 and ending with residue 288, 289, 290, 291, 292, 293, 294, 295, 296, 297, or 298 of SEQ ID NO: 2. In one embodiment, a first IL-12 p40 domain (p40N) encoded by polynucleotides of the invention comprises, or alternatively consists of, amino acid residues 23 to 293 of SEQ ID NO: 2.

The optional first peptide linker (ii) may be any suitable peptide linker that allows folding of the scIL-12 polypeptide into a functional protein. In certain embodiments, the optional first peptide linker encoded by polynucleotides of the invention consists of 10 or fewer amino acids. In specific embodiments, the first peptide linker consists of 1, 2, 3, 4, 5, 6, 7, 8, 9, or 10 amino acids. In specific embodiments, the first peptide linker comprises any sequence and combination of one or more amino acids selected from: Glycine (Gly); Serine (Ser); Alanine (Ala); Threonine (Thr); and, Proline (Pro). In a preferred embodiment, the first peptide linker is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the first peptide linker is absent.

In certain embodiments, the IL-12 p35 domain (iii) encoded by polynucleotides of the invention is a mature IL-12 p35 subunit, lacking a signal peptide. IL-12 p35 polynucleotides for use in the invention include the human IL-12 p35 nucleic acid sequence of SEQ ID NO: 3 and the murine IL-12 p35 nucleic acid sequence of SEQ ID NO: 7. Additional, non-limiting examples of polynucleotides encoding IL-12 p35 subunits are available in public sequence databases, including but not limited to AF101062.1 (human), NM_000882.3 (human), NG_033022.1 (human), NM_001159424.1 (mouse), NM_008351.2 (mouse), NM_001009833 (cat), NM_001082511.1 (horse), NM_001003293.1 (dog).

It is understood that the specific cleavage site of a signal peptide may vary by 1, 2, 3 or more residues. Accordingly, IL-12 p35 domains encoded by polynucleotides of the invention include the predicted mature sequence comprising, or alternatively consisting of, residues 57 to 253 of SEQ ID NO: 4 as well as mature sequences comprising, or alternatively consisting of, amino acids 52 to 253, 53 to 253, 54 to 253, 55 to 253, 56 to 253, 58 to 253, 59 to 253, 60 to 253, 61 to 263 and 62 to 253 of SEQ ID NO: 4.

Suitable IL-12 p35 domains encoded by polynucleotides of the invention may be truncated at the C-terminus by one or more amino acid residues. Therefore, in additional embodiments the IL-12 p35 domain encoded by polynucleotides of the invention comprise, or alternatively consist of, a fragment of SEQ ID NO: 4 beginning with residue 52, 53, 54, 55, 56, 57, 5 8, 59, 60, 61, or 62 of SEQ ID NO: 4 and ending with residue 247, 248, 249, 250, 251, 252, or 253 of SEQ ID NO: 4.

The optional second peptide linker (iv) may be any suitable peptide linker that allows folding of the scIL-12 polypeptide into a functional protein. In certain embodiments, the optional second peptide linker encoded by polynucleotides of the invention consists of 10 or fewer amino acids. In specific embodiments, the second peptide linker consists of 1, 2, 3, 4, 5, 6, 7, 8, 9, or 10 amino acids. In specific embodiments, the second peptide linker comprises any sequence and combination of one or more amino acids selected from: Glycine (Gly); Serine (Ser); Alanine (Ala); Threonine (Thr); and, Proline (Pro). In a preferred embodiment, the second peptide linker is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the second peptide linker is absent. In a preferred embodiment, the first and second peptide linkers consist of 10, 9, 8, 7 or fewer amino acid residues combined.

In certain embodiments, the second IL-12 p40 domain (also referred to herein as p40C) encoded by polynucleotides of the invention is a C-terminal fragment of an IL-12 p40 subunit. C-terminal fragments of IL-12 p40 encoded by polynucleotides of the invention and suitable as a second IL-12 p40 domain (p40C) comprise, or alternatively consist of, amino acids 289 to 328, 290 to 328, 291 to 328, 292 to 328, 293 to 328, 294 to 328, 295 to 328, 296 to 328, 297 to 328, 298 to 328, and 299 to 328 of SEQ ID NO: 2.

Suitable second IL-12 p40 domains (p40C) encoded by polynucleotides of the invention may be truncated at the C-terminus by one or more amino acid residues. Accordingly, in additional embodiments the second IL-12 p40 domain (p40C) encoded by polynucleotides of the invention comprises, or alternatively consists of, a fragment of SEQ ID NO: 2 beginning with residue 289, 290, 291, 292, 293, 294, 295, 296, 297, 298, or 299 of SEQ ID NO: 2 and ending with residue 322, 323, 324, 325, 326, 327, or 328 of SEQ ID NO: 2.

The full-length sequence of a polynucleotide encoding a preferred scIL-12 polypeptide of the invention is presented herein as SEQ ID NO: 9. The full-length sequence encodes a predicted signal peptide at nucleic acids 1 to 66 of SEQ ID NO: 9, and a mature scIL-12 polypeptide at nucleic acids 67 to 1599 of SEQ ID NO: 9.

Thus, a first subject of the invention relates to an isolated polynucleotide encoding a novel scIL-12 polypeptide. In a specific embodiment, the isolated polynucleotide comprises a nucleic acid sequence selected from the group consisting of SEQ ID NO: 9 and nucleic acids 67 to 1599 of SEQ ID NO: 9. In a specific embodiment, the isolated polynucleotide further comprises a region permitting expression of the polypeptide in a host cell.

The present invention also relates to an isolated polynucleotide encoding a scIL-12 polypeptide comprising an amino acid sequence selected from the group consisting of SEQ ID NO: 10 and amino acids 23 to 533 of SEQ ID NO: 10.

The invention also provides polynucleotides encoding variants of the scIL-12 polypeptides of the invention. In a preferred embodiment the polynucleotides of the invention encode a scIL-12 variant polypeptide at least 80%, at least 85%, at least 90%, at least 95%, at least 97%, at least 98%, or at least 99% identical to the full-length or mature amino acid sequence of SEQ ID NO: 10, where the variant polypeptide exhibits at least one IL-12 activity, such as induction of IFN-gamma secretion from NK cells. Such IL-12 activities are readily determined using assays known in the art, such as the assays described in Example 8 of U.S. Pat. No. 5,457,038, which is incorporated herein by reference.

Due to the degeneracy of nucleotide coding sequences, other polynucleotides that encode substantially the same amino acid sequence as a scIL-12 polynucleotide disclosed herein, including an amino acid sequence that contains a single amino acid variant, may be used in the practice of the present invention. These include but are not limited to allelic genes, homologous genes from other species, and nucleotide sequences comprising all or portions of a scIL-12 polynucleotide that are altered by the substitution of different codons that encode the same amino acid residue within the sequence, thus producing a silent change. Likewise, the scIL-12 derivatives of the invention include, but are not limited to, those comprising, as a primary amino acid sequence, all or part of the amino acid sequence of a scIL-12 polypeptide including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations can be produced by various methods known in the art (see Sambrook et al., 1989, infra) and are not expected to affect apparent molecular weight as determined by polyacrylamide gel electrophoresis, or isoelectric point.

The present invention also relates to an isolated scIL-12 polypeptide encoded by a polynucleotide according to the invention.

### Single Chain IL-12 Polypeptides

The present invention provides novel scIL-12 polypeptides, including full length and mature scIL-12 polypeptides.

Thus, the invention relates to isolated scIL-12 polypeptides. In a specific embodiment, the invention provides a scIL-12 polypeptide comprising, from N- to C-terminus:

(i) a first IL-12 p40 domain (p40N),

(ii) an optional first peptide linker,

(iii) an IL-12 p35 domain,

(iv) an optional second peptide linker, and

(v) a second IL-12 p40 domain (p40C).

In certain embodiments, the first IL-12 p40 do main (p40N) is an N-terminal fragment of an IL-12 p40 subunit. IL-12 p40 polypeptides for use in the invention include the human IL-12 p40 amino acid sequence of SEQ ID NO: 2 and the murine IL-12 p40 amino acid sequence of SEQ ID NO: 6. Additional, non-limiting examples of IL-12 p40 subunits are available in public sequence databases, including but not limited to Genbank Accession Nos. P29460.1 (human), AAD56386.1 (human), NP_005526.1 (human), NP_714912.1 (human), Q28268.1 (dog), NP_001003292.1 (dog), NP_032378.1 (mouse), NP_001152896.1 (mouse), NP_032377.1 (mouse).

N-terminal fragments of IL-12 p40 suitable as a first IL-12 p40 domain (p40N) include, but are not limited to, polypeptides comprising, or alternatively consisting of, amino acids 1 to 288, 1 to 289, 1 to 290, 1 to 291, 1 to 292, 1 to 293, 1 to 294, 1 to 295, 1 to 296, 1 to 297, and 1 to 298 of SEQ ID NO: 2. A preferred first IL-12 p40 domain (p40N) comprises, or alternatively consists of, amino acids 1 to 293 of SEQ ID NO: 2.

N-terminal fragments of IL-12 p40 suitable as a first IL-12 p40 domain (p40N) may lack a signal sequence. Therefore, in additional embodiments the first IL-12 p40 domain (p40N) comprises, or alternatively consists of, a fragment of SEQ ID NO: 2 beginning with residue 18, 19, 20, 21, 22, 23, 24, 25, 26, 27 or 28 of SEQ ID NO: 2 and ending with residue 288, 289, 290, 291, 292, 293, 294, 295, 296, 297, or 298. In one embodiment, the first IL-12 p40 domain (p40N) comprises, or alternatively consists of, amino acid residues 23 to 293 of SEQ ID NO: 2.

The optional first peptide linker (ii) may be any suitable peptide linker that allows folding of the scIL-12 polypeptide into a functional protein. In certain embodiments, the optional first peptide linker consists of 10 or fewer amino acids. In specific embodiments, the first peptide linker consists of 1, 2, 3, 4, 5, 6, 7, 8, 9, or 10 amino acids. In specific embodiments, the first peptide linker comprises any sequence and combination of one or more amino acids selected from: Glycine (Gly); Serine (Ser); Alanine (Ala); Threonine (Thr); and, Proline (Pro). In a preferred embodiment, the first peptide linker is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the first peptide linker is absent.

In certain embodiments, the IL-12 p35 domain (iii) is a mature IL-12 p35 subunit, lacking a signal peptide. IL-12 p35 polypeptides for use in the invention include the human IL-12 p35 amino acid sequence of SEQ ID NO: 4 and the murine IL-12p35 amino acid sequence of SEQ ID NO: 8. Additional, non-limiting examples of IL-12 p35 subunits are available in public sequence databases, including but not limited to Genbank Accession Nos. AAB32758.1 (cat), NP_001003293 (dog), NP_001075980.1 (horse), NP_000873.2 (human), AAD56385.1 (human), NP_001152896.1 (mouse), and NP_032377.1 (mouse).

It is understood that the specific cleavage site of a signal peptide may vary by 1, 2, 3 or more residues. Accordingly, in certain embodiments, mature p35 polypeptides of the invention include the predicted mature sequence consisting of residues 57 to 253 of SEQ ID NO: 4 as well as mature sequences consisting of amino acids 52 to 253, 53 to 253, 54 to 253, 55 to 253, 56 to 253, 58 to 253, 59 to 253, 60 to 253, 61 to 263 and 62 to 253 of SEQ ID NO: 4.

Suitable IL-12 p35 domains may be truncated at the C-terminus by one or more amino acid residues. Therefore, in additional embodiments the IL-12 p35 domain comprises, or alternatively consists of, a fragment of SEQ ID NO: 4 beginning with residue 52, 53, 54, 55, 56, 57, 58, 59, 60, 61, or 62 of SEQ ID NO: 4 and ending with residue 247, 248, 249, 250, 251, 252, or 253 of SEQ ID NO: 4.

The optional second peptide linker (iv) may be any suitable peptide linker that allows folding of the scIL-12 polypeptide into a functional protein. In certain embodiments, the optional second peptide linker consists of 10 or fewer amino acids. In specific embodiments, the second peptide linker consists of 1, 2, 3, 4, 5, 6, 7, 8, 9, or 10 amino acids. In specific embodiments, the second peptide linker comprises any sequence and combination of one or more amino acids selected from: Glycine (Gly); Serine (Ser); Alanine (Ala); Threonine (Thr); and, Proline (Pro). In preferred embodiments, the second peptide linker is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the second peptide linker is absent. In a preferred embodiment, the first and second peptide linkers consist of 10 or fewer amino acid residues combined.

In certain embodiments, the second IL-12 p40 domain (p40C) is a C-terminal fragment of an IL-12 p40 subunit. C-terminal fragments of p40 suitable as a second IL-12 p40 domain (p40C) comprise, or alternatively consist of, amino acids 289 to 328, 290 to 329, 291 to 328, 292 to 328, 293 to 328, 294 to 328, 295 to 328, 296 to 328, 297 to 328, 298 to 328, and 299 to 328 of SEQ ID NO: 2.

Suitable second IL-12 p40 domains (p40C) may be truncated at the C-terminus by one or more amino acid residues. Therefore, in additional embodiments the second IL-12 p40 domain (p40C) comprises, or alternatively consists of, a fragment of SEQ ID NO: 2 beginning with residue 289, 290, 291, 292, 293, 294, 295, 296, 297, 298, or 299 of SEQ ID NO: 2 and ending with residue 322, 323, 324, 325, 326, 327, or 328 of SEQ ID NO: 2.

The full-length sequence of a representative scIL-12 polypeptide of the invention is presented herein as SEQ ID NO: 10. The full-length sequence contains a predicted signal peptide at amino acids 1 to 22 of SEQ ID NO: 10, and a mature scIL-12 polypeptide at amino acids 23 to 533 of SEQ ID NO: 10.

In another specific embodiment, the scIL-12 polypeptide is encoded by a polynucleotide comprising a nucleic acid sequence selected from the group consisting of SEQ ID NO: 9 and nucleotides 67 to 1599 of SEQ ID NO: 9.

Thus, a first subject of the invention relates to an isolated scIL-12 polypeptide. In a specific embodiment, the isolated polypeptide comprises an amino acid sequence selected from the group consisting of SEQ ID NO: 10 and amino acids 23 to 533 of SEQ ID NO: 10.

One of skill in the art is able to produce other polynucleotides to encode the polypeptides of the invention, by making use of the present invention and the degeneracy or non-universality of the genetic code as described herein.

Additional embodiments of the present invention include functional fragments of a scIL-12 polypeptide, or fusion proteins comprising a scIL-12 polypeptide of the present invention fused to second polypeptide comprising a heterologous, or normally non-contiguous, protein domain. Preferably, the second polypeptide is a targeting polypeptide such as an antibody, including single chain antibodies or antibody fragments. Thus, the invention provides a ScIL-12 polypeptide fused at its N- or C-terminus to a second polypeptide, preferably to an antibody, an antibody fragment, or a single chain antibody.

The invention also provides variants of the scIL-12 polypeptides of the invention. In certain embodiments a scIL-12 variant polypeptide is at least 80%, at least 85%, at least 90%, at or at least 95%, at least 97%, at least 98%, or at least 99% identical to the full-length or mature amino acid sequence of SEQ ID NO: 10, where the variant polypeptide exhibits at least one IL-12 activity, such as induction of IFN-gamma secretion from NK cells. Such IL-12 activities are readily determined using assays known in the art, such as the assays described in Example 8 of U.S. Pat. No. 5,457,038, which is incorporated herein by reference.

The present invention also relates to compositions comprising an isolated polypeptide according to the invention.

### Compositions

The present invention also relates to compositions comprising the scIL-12 polynucleotides or polypeptides according to the invention. Such compositions may comprise a scIL-12 polypeptide or a polynucleotide encoding a scIL-12 polypeptide, as defined above, and an acceptable carrier or vehicle. The compositions of the invention are particularly suitable for formulation of biological material for use in therapeutic administration. Thus, in one embodiment, the composition comprises a polynucleotide encoding a scIL-12 polypeptide. In another embodiment, the composition comprises a scIL-12 polypeptide according to the invention.

The phrase “acceptable” refers to molecular entities and compositions that are physiologically tolerable to the cell or organism when administered. The term “carrier” refers to a diluent, adjuvant, excipient, or vehicle with which the composition is administered. Such carriers can be sterile liquids, such as water and oils, including those of petroleum, animal, vegetable or synthetic origin, such as peanut oil, soybean oil, mineral oil, sesame oil and the like. Examples of acceptable carriers are saline, buffered saline, isotonic saline (e.g., monosodium or disodium phosphate, sodium, potassium, calcium or magnesium chloride, or mixtures of such salts), Ringer's solution, dextrose, water, sterile water, glycerol, ethanol, and combinations thereof 1,3 -butanediol and sterile fixed oils are conveniently employed as solvents or suspending media. Any bland fixed oil can be employed including synthetic mono- or di-glycerides. Fatty acids such as olcic acid also find use in the preparation of injectables. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. Pharmaceutical compositions of the invention may be formulated for the purpose of topical, oral, parenteral, intranasal, intravenous, intramuscular, intratumoral, subcutaneous, intraocular, and the like, administration.

Preferably, the compositions comprise an acceptable vehicle for an injectable formulation. This vehicle can be, in particular, a sterile, isotonic saline solution (monosodium or disodium phosphate, sodium, potassium, calcium or magnesium chloride, and the like, or mixtures of such salts), or dry, in particular lyophilized, compositions which, on addition, as appropriate, of sterilized water or of physiological saline, enable injectable solutions to be formed. The preferred sterile injectable preparations can be a solution or suspension in a nontoxic parenterally acceptable solvent or diluent.

In yet another embodiment, a composition comprising a scIL-12 polypeptide, or polynucleotide encoding the polypeptide, can be delivered in a controlled release system. For example, the polynucleotide or polypeptide may be administered using intravenous infusion, an implantable osmotic pump, a transdermal patch, liposomes, or other modes of administration. Other controlled release systems are discussed in the review by Langer [Science 249:1527-1533 (1990)].

### Expression of Single Chain IL-12 Polypeptides

With the sequence of the scIL-12 polypeptides and the polynucleotides encoding them, large quantities of scIL-12 polypeptides may be prepared. By the appropriate expression of vectors in cells, high efficiency production may be achieved. Thereafter, standard purification methods may be used, such as ammonium sulfate precipitations, column chromatography, electrophoresis, centrifugation, crystallization and others. See various volumes of Methods in Enzymology for techniques typically used for protein purification. Alternatively, in some embodiments high efficiency of production is unnecessary, but the presence of a known inducing protein within a carefully engineered expression system is quite valuable. Typically, the expression system will be a cell, but an in vitro expression system may also be constructed.

A polynucleotide encoding a scIL-12, or fragment, derivative or analog thereof, or a functionally active derivative, including a chimeric protein, thereof, can be inserted into an appropriate expression vector, i.e., a vector which comprises the necessary elements for the transcription and translation of the inserted protein-coding sequence. A polynucleotide of the invention is operationally linked with a transcriptional control sequence in an expression vector. An expression vector also preferably includes a replication origin.

The isolated polynucleotides of the invention may be inserted into any appropriate cloning vector. A large number of vector-host systems known in the art may be used. Possible vectors include, but are not limited to, plasmids or modified viruses, but the vector system must be compatible with the host cell used. Examples of vectors include, but are not limited to, Escherichia coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the polynucleotide into a cloning vector that has complementary cohesive termini. However, if the complementary restriction sites used to fragment the polynucleotide are not present in the cloning vector, the ends of the polynucleotide molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Preferably, the cloned gene is contained on a shuttle vector plasmid, which provides for expansion in a cloning cell, e.g., E. coli, and purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2 μ plasmid.

In addition, the present invention relates to an expression vector comprising a polynucleotide according the invention, operatively linked to a transcription regulatory element. In one embodiment, the polynucleotide is operatively linked with an expression control sequence permitting expression of the scIL-12 polypeptide in an expression competent host cell. The expression control sequence may comprise a promoter that is functional in the host cell in which expression is desired. The vector may be a plasmid DNA molecule or a viral vector. In certain embodiments, viral vectors include, without limitation, retrovirus, adenovirus, adeno-associated virus (AAV), herpes virus, and vaccinia virus. The invention further relates to a replication defective recombinant virus comprising in its genome, a polynucleotide according to the invention. Thus, the present invention also relates to an isolated host cell comprising such an expression vector, wherein the transcription regulatory element is operative in the host cell.

The desired genes will be inserted into any of a wide selection of expression vectors. The selection of an appropriate vector and cell line depends upon the constraints of the desired product. Typical expression vectors are described in Sambrook et al. (1989). Suitable cell lines may be selected from a depository, such as the ATCC. See, ATCC Catalogue of Cell Lines and Hybridomas (6th ed.) (1988); ATCC Cell Lines, Viruses, and Antisera, each of which is hereby incorporated herein by reference. The vectors are introduced to the desired cells by standard transformation or transfection procedures as described, for instance, in Sambrook et al. (1989).

Fusion proteins will typically be made by either recombinant nucleic acid methods or by synthetic polypeptide methods. Techniques for nucleic acid manipulation are described generally, for example, in Sambrook et al. (1989), Molecular Cloning: A Laboratory Manual (2d ed.), Vols. 1-3, Cold Spring Harbor Laboratory, which are incorporated herein by reference. Techniques for synthesis of polypeptides are described, for example, in Merrifield, J. Amer. Chem. Soc. 85:2149-2156 (1963).

Once a particular recombinant DNA molecule is identified and isolated, any of multiple methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: hum an or animal viruses such as vaccinia virus, adenovirus, or adeno-associated virus (AAV); insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Different host cells have characteristic and specific mechanisms for the translational and post-translational processing and modification of proteins. Appropriate cell lines or host systems can be chosen to ensure the desired modification and processing of the foreign protein expressed. Expression in yeast can produce a biologically active product. Expression in eukaryotic cells can increase the likelihood of “native” folding. Moreover, expression in mammalian cells can provide a tool for reconstituting, or constituting, scIL-12 activity. Furthermore, different vector/host expression systems may affect processing reactions, such as proteolytic cleavages, to a different extent.

Vectors are introduced into the desired host cells by methods known in the art, e.g., transfection, electroporation, microinjection, transduction, cell fusion, DEAE dextran, calcium phosphate precipitation, lipofection (lysosome fusion), particle bombardment, use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Vectors and Gene Expression Cassettes Comprising scIL-12 Polynucleotides

The present invention also relates to a vector comprising a polynucleotide encoding a scIL-12 polypeptide according to the invention. The present invention also provides a gene expression cassette comprising a polynucleotide encoding a scIL-12 polypeptide according to the invention. The polynucleotides of the invention, where appropriate incorporated in vectors or gene expression cassettes, and the compositions comprising them, are useful for enhancing immune system function, for example as vaccine adjuvants and in combination with other immunomodulators and/or small molecule pharmaceuticals in the treatment of infections and cancer. They may be used for the transfer and expression of genes in vitro or in vivo in any type of cell or tissue. The transformation can, moreover, be targeted (transfer to a particular tissue can, in particular, be determined by the choice of a vector, and expression by the choice of a particular promoter). The polynucleotides and vectors of the invention are advantageously used for the production in vivo of scIL-12 polypeptides of the invention.

The polynucleotides encoding the scIL-12 polypeptides of the invention may be used in a plasmid vector. Preferably, an expression control sequence is operably linked to the scIL-12 polynucleotide coding sequence for expression of the scIL-12 polypeptide. The expression control sequence may be any enhancer, response element, or promoter system in vectors capable of transforming or transfecting a host cell. Once the vector has been incorporated into the appropriate host, the host, depending on t he use, will be maintained under conditions suitable for high level expression of the polynucleotides.

Polynucleotides will normally be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference).

Escherichia coli is one prokaryotic host useful for cloning the polynucleotides of the present invention. Other microbial hosts suitable for use include, without limitation, bacilli, such as Bacillus subtilis, and other enterobacteriaceae, such as Salmonella, Serratia, and various Pseudomonas species.

Other eukaryotic cells may be used, including, without limitation, yeast cells, insect tissue culture cells, avian cells or the like. Preferably, mammalian tissue cell culture will be used to produce the polypeptides of the present invention (see, Winnacker, From Genes to Clones, VCH Publishers, N.Y. (1987), which is incorporated herein by reference).

Expression vectors may also include, without limitation, expression control sequences, such as an origin of replication, a promoter, an enhancer, a response element, and necessary processing information sites, such as ribosome-binding sites, RNA splice sites, polyadenylation sites, and transcriptional terminator sequences. Preferably, the enhancers or promoters will be those naturally associated with genes encoding the IL-12 subunits p40 and p35, although it will be understood that in many cases others will be equally or more appropriate. In further embodiments, expression control sequences are enhancers or promoters derived from viruses, such as SV40, Adenovirus, Bovine Papilloma Virus, and the like.

The vectors comprising the polynucleotides of the present invention can be transferred into the host cell by well-known methods, which vary depending on the type of cellular host. For example, calcium chloride transfection is commonly utilized for procaryotic cells, whereas calcium phosphate treatment may be used for other cellular hosts. (See, generally, Sambrook et al. (1989), Molecular Cloning: A Laboratory Manual (2d ed.), Cold Spring Harbor Press, which is incorporated herein by reference.) The term “transformed cell” is meant to also include the progeny of a transformed cell.

Potential host-vector systems include but are not limited to mammalian cell systems infected with virus (e.g., vaccinia virus, adenovirus, adeno-associated virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant scIL-12 protein of the invention, or functional fragment, derivative, chimeric construct, or analog thereof, may be expressed chromosomally, after integration of the coding sequence by recombination. In this regard, any of a number of amplification systems may be used to achieve high levels of stable gene expression (See Sambrook et al., 1989, supra).

The cell containing the recombinant vector comprising the scIL-12 polynucleotide is cultured in an appropriate cell culture medium under conditions that provide for expression of the scIL-12 polypeptide by the cell. Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

A polynucleotide encoding a scIL-12 polypeptide may be operably linked and controlled by any regulatory region, i.e., promoter/enhancer element known in the art, but these regulatory elements must be functional in the host cell selected for expression. The regulatory regions may comprise a promoter region for functional transcription in the host cell, as well as a region situated 3′ of the gene of interest, and which specifies a signal for termination of transcription and a polyadenylation site. All these elements constitute an expression cassette.

Expression vectors comprising a polynucleotide encoding a scIL-12 polypeptide of the invention can be identified by five general approaches: (a) PCR amplification of the desired plasmid DNA or specific mRNA, (b) nucleic acid hybridization, (c) presence or absence of selection marker gene functions, (d) analyses with appropriate restriction endonucleases, and (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding a scIL-12 polypeptide is inserted within the “selection marker” gene sequence of the vector, recombinants comprising the scIL-12 nucleic acid insert can be identified by the absence of the gene function. In the fourth approach, recombinant expression vectors are identified by digestion with appropriate restriction enzymes. In the fifth approach, recombinant expression vectors can be identified by assaying for the activity, biochemical, or immunological characteristics of the gene product expressed by the recombinant, provided that the expressed protein assumes a functionally active conformation.

A wide variety of host/expression vector combinations may be employed in expressing the DNA sequences of this invention. Useful expression vectors, for example, may consist of segments of chromosomal, non-chromosomal and synthetic DNA sequences. Suitable vectors include but are not limited to derivatives of SV40 and known bacterial plasmids, e.g., E. coli plasmids col E1, pCR1, pBR322, pMal-C2, pET, pGEX (Smith et al., 1988, Gene 67:31-40), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

The present invention also provides a gene expression cassette that is capable of being expressed in a host cell, wherein the gene expression cassette comprises a polynucleotide that encodes a scIL-12 polypeptide according to the invention. Thus, Applicants' invention also provides novel gene expression cassettes useful in a scIL-12 expression system.

Gene expression cassettes of the invention may include a gene switch to allow the regulation of gene expression by addition or removal of a specific ligand. In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands glucocorticoid, estrogen, progestin, retinoid, ecdysone, and analogs and mimetics thereof); rTTA activated by tetracycline; Biotin-based switch systems; FKBP/rapamycin switch systems; cumate switch systems; riboswitch systems; among others.

In one aspect of the invention, the gene switch is an EcR-based gene switch. Examples of such systems include, without limitation, the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/U52002/005706 (WO 2002/066614); U.S. Pat. Nos. 7,531,326; 8,236,556; 8,598,409; PCT/US2002/005090 (WO 2002/066612); U.S. application Ser. No. 10/468,193 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. application Ser. No. 14/001,943 (U.S. Pub. No. [Pending]), each of which is incorporated by reference in its entirety.

In another aspect of the invention, the gene switch is based on heterodimerization of FK506 binding protein (FKBP) with FKBP rapamycin associated protein (FRAP) and is regulated through rapamycin or its non-immunosuppressive analogs. Examples of such systems include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595.

In another aspect of the invention, gene expression cassettes of the invention incorporate a cumate switch system, which works through the CymR repressor that binds the cumate operator sequences with high affinity. (SparQ™ Cumate Switch, System Biosciences, Inc.) The repression is alleviated through the addition of cumate, a non-toxic small molecule that binds to CymR. This system has a dynamic inducibility, can be finely tuned and is reversible and inducible.

In another aspect of the invention, gene expression cassettes of the invention incorporate a riboswitch, which is a regulatory segment of a messenger RNA molecule that binds an effector, resulting in a change in production of the proteins encoded by the mRNA. An mRNA that contains a riboswitch is directly involved in regulating its own activity in response to the concentrations of its effector molecule. Effectors can be metabolites derived from purine/pyrimidine, amino acid, vitamin, or other small molecule co-factors. These effectors act as ligands for the riboswitch sensor, or aptamer. Breaker, RR. Mol Cell. (2011) 43(6):867-79.

In another aspect of the invention, gene expression cassettes of the invention incorporate the biotin-based gene switch system, in which the bacterial repressor protein TetR is fused to streptavidin, which interacts with the synthetic biotinylation signal AVITAG that is fused to VP 16 to activate gene expression. Biotinylation of the AVITAG peptide is regulated by a bacterial biotin ligase BirA, thus enabling ligand responsiveness. Weber et al. (2007) Proc. Natl. Acad. Sci. U.S.A. 104, 2643-2648; Weber et al. (2009) Metabolic Engineering, 11(2):117-124.

Additional gene switch systems appropriate for use in the instant invention are well known in the art, including but not limited to those described in Auslander and Fussenegger, Trends in Biotechnology (2012), 31(3):155-168, incorporated herein by reference.

Examples of ligands for use in gene switch systems include, without limitation, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; arnidoketones such as those described in U.S. Published Application No. 2004/0049037; each of which is incorporated herein by reference and other similar materials including 3,5 -di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, and the like. Examples of diacylhydrazine ligands useful in the present invention include RG-115819 (3,5-Dimethyl-benzoic acid N-(1 -ethyl-2,2-dimethyl-propyl)-N′-(2-methyl-3 -methoxy-benzoyl)-hydrazide-), RG-115932 ((R)-3,5-Dimethyl-benzoic acid N-(1 -tert-butyl-butyl)-N′-(2-ethyl-3 -methoxy-benzoyl)-hydrazide), and RG-115830 (3,5 -Dimethyl-benzoic acid N-(1 -tert-butyl-butyl)-N′-(2-ethyl-3 -methoxy-benzoyl)-hydrazide). See, e.g., U.S. patent application Ser. No. 12/155,111, and PCT Appl. No. PCT/US2008/006757, both of which are incorporated herein by reference in their entireties.

### Antibodies to Single Chain IL-12 Polypeptides

According to the invention, a scIL-12 polypeptide produced recombinantly or by chemical synthesis, and fragments or other derivatives or analogs thereof, including fusion proteins, may be used as an antigen or immunogen to generate antibodies. Preferably, the antibodies specifically bind scIL-12 polypeptides, but do not bind native IL-12 polypeptides. More preferably, the antibodies specifically bind a scIL-12 polypeptide, but do not bind other cytokine polypeptides.

In another embodiment, the invention relates to an antibody which specifically binds an antigenic peptide comprising a fragment of a scIL-12 polypeptide according to the invention as described above. The antibody may be polyclonal or monoclonal and may be produced by in vitro or in vivo techniques.

The antibodies of the invention possess specificity for binding to particular scIL-12 polypeptides. Thus, reagents for determining qualitative or quantitative presence of these or homologous polypeptides may be produced. Alternatively, these antibodies may be used to separate or purify scIL-12 polypeptides.

For production of polyclonal antibodies, an appropriate target immune system is selected, typically a mouse or rabbit. The substantially purified antigen is presented to the immune system in a fashion determined by methods appropriate for the animal and other parameters well known to immunologists. Typical sites for injection are in the footpads, intramuscularly, intraperitoneally, or intradermally. Of course, another species may be substituted for a mouse or rabbit.

An immunological response is usually assayed with an immunoassay. Normally such immunoassays involve some purification of a source of antigen, for example, produced by the same cells and in the same fashion as the antigen was produced. The immunoassay may be a radioimmunoassay, an enzyme-linked assay (ELISA), a fluorescent assay, or any of many other choices, most of which are functionally equivalent but may exhibit advantages under specific conditions.

Monoclonal antibodies with high affinities are typically made by standard procedures as described, e.g., in Harlow and Lane (1988), Antibodies: A Laboratory Manual, Cold Spring Harbor Laboratory; or Goding (1986), Monoclonal Antibodies: Principles and Practice (2d ed) Academic Press, New York, which are hereby incorporated herein by reference. Briefly, appropriate animals will be selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone are tested for their production of an appropriate antibody specific for the desired region of the antigen.

Other suitable techniques involve in vitro exposure of lymphocytes to the antigenic polypeptides or alternatively to selection of libraries of antibodies in phage or similar vectors. See, Huse et al., (1989) “Generation of a Large Combinatorial Library of the Immunoglobulin Repertoire in Phage Lambda,” Science 246:1275-1281, hereby incorporated herein by reference.

The polypeptides and antibodies of the present invention may be used with or without modification. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include, without limitation, radionuclides, enzymes, substrates, cofactors, inhibitors, fluorescence, chemiluminescence, magnetic particles and the like. Patents, teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly, U.S. Pat. No. 4,816,567.

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10 amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

Such antibodies include but are not limited to polyclonal, monoclonal, chimeric, single chain, Fab fragments, and an Fab expression library. The scIL-12 antibodies of the invention may be cross reactive, e.g., they may recognize scIL-12 polypeptides derived from different species. Polyclonal antibodies have greater likelihood of cross reactivity. Alternatively, an antibody of the invention may be specific for a single form of scIL-12 polyptide, such as a human scIL-12 polypeptide. Preferably, such an antibody is specific for human scIL-12.

Various procedures known in the art may be used for the production of polyclonal antibodies. For the production of antibody, various host animals can be immunized by injection with a scIL-12 polypeptide, or a derivative (e.g., fragment or fusion protein) thereof, including but not limited to rabbits, mice, rats, sheep, goats, etc. In one embodiment, the scIL-12 polypeptide or fragment thereof can be conjugated to an immunogenic carrier, e.g., bovine serum albumin (BSA) or keyhole limpet hemocyanin (KLH). Various adjuvants may be used to increase the immunological response, depending on t he host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward a scIL-12 polypeptide, or fragment, analog, or derivative thereof, any technique that provides for the production of antibody molecules by continuous cell lines in culture may be used. These include but are not limited to the hybridoma technique originally developed by Kohler and Milstein [Nature 256:495-497 (1975)], as well as the trioma technique, the human B-cell hybridoma technique [Kozbor et al., Immunology Today 4:72 1983); Cote et al., Proc. Natl. Acad. Sci. U.S.A. 80:2026-2030 (1983)], and the EBV-hybridoma technique to produce human monoclonal antibodies [Cole et al., in Monoclonal Antibodies and Cancer Therapy, Alan R. Liss, Inc., pp. 77 -96 (1985)]. In an additional embodiment of the invention, monoclonal antibodies can be produced in germ-free animals [International Patent Publication No. WO 89/12690, published 28 December 1989]. In fact, according to the invention, techniques developed for the production of “chimeric antibodies” [Morrison et al., J. Bacteriol. 159:870 (1984); Neuberger et al., Nature 312:604-608 (1984); Takeda et al., Nature 314:452-454 (1985)] by splicing the genes from a mouse antibody molecule specific for a scIL-12 polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain Fv (scFv) antibodies [U.S. Pat. Nos. 5,476,786 and 5,132,405 to Huston; U.S. Pat. No. 4,946,778] can be adapted to produce scIL-12 polypeptide-specific single chain antibodies. An additional embodiment of the invention utilizes the techniques described for the construction of Fab expression libraries [Huse et al., Science 246:1275-1281 (1989)] to allow rapid and easy identification of monoclonal Fab fragments with the desired specificity for a scIL-12 polypeptide, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a scIL-12 polypeptide, one may assay generated hybridomas for a product which binds to a scIL-12 polypeptide fragment containing such epitope.

The foregoing antibodies can be used in methods known in the art relating to the localization and activity of a scIL-12 polypeptide, e.g., for western blotting, imaging a scIL-12 polypeptide in situ, measuring levels thereof in appropriate physiological samples, etc. using any of the detection techniques mentioned above or known in the art.

### Uses of Single Chain IL-12 Polynucleotides and Polypeptides

The scIL-12 polypeptides and polynucleotides of the present invention have a variety of utilities. For example, the polynucleotides and polypeptides of the invention are useful in the treatment of diseases in which stimulation of immune function might be beneficial. In specific embodiments, the scIL-12 polypeptides and polynucleotides of the present invention are useful for the treatment of disease states responsive to the enhanced presence of gamma interferon; for the treatment of viral, bacterial, protozoan and parasitic infections; and for the treatment of proliferative disorders such as cancer. The scIL-12 polynucleotides and polypeptides of the invention are also useful as vaccine adjuvants.

### Methods of Inducing IFN-gamma Production

The scIL-12 polypeptide and polynucleotide compositions of the invention are useful for inducing the production of IFN-gamma in a patient in need thereof. Pathological states which benefit from IFN-gamma induction may result from disease, exposure to radiation or drugs, and include for example but without limitation, leukopenia, bacterial and viral infections, anemia, B cell or T cell deficiencies including immune cell or hematopoietic cell deficiency following a bone marrow transplantation.

### Methods of Treating Infections

The scIL-12 polypeptide and polynucleotide compositions according to the present invention can be used in the treatment of viral infections, including without limitation, HIV, Hepatitis A, Hepatitis B, Hepatitis C, rabies virus, poliovirus, influenza virus, meningitis virus, measles virus, mumps virus, rubella, pertussis, encephalitis virus, papilloma virus, yellow fever virus, respiratory syncytial virus, parvovirus, chikungunya virus, haemorrhagic fever viruses, Klebsiella, and Herpes viruses, particularly, varicella, cytomegalovirus and Epstein-Barr virus infection, among others.

The scIL-12 polypeptide and polynucleotide compositions according to the present invention can be used in the treatment of bacterial infections, including, without limitation, leprosy, tuberculosis, Yersinia pestis, Typhoid fever, pneumococcal bacterial infections, tetanus and anthrax, among others.

The scIL-12 polypeptide and polynucleotide compositions according to the present invention can also be used in the treatment of parasitic infections, such as, but not limited to, leishmaniasis and malaria, among others; and protozoan infections, such as, but not limited to, T. cruzii) or helminths, such as Schistosoma.

### Methods of Use as a Vaccine Adjuvant

The scIL-12 polypeptide and polynucleotide compositions are useful as vaccine adjuvants. B y “adjuvant” is meant a substance which enhances the immune response when administered together with an immunogen or antigen.

The scIL-12 polypeptide and polynucleotide compositions of the invention are useful for enhancing the immune response to viral vaccines, including without limitation, HIV, Hepatitis A, Hepatitis B, Hepatitis C, rabies virus, poliovirus, influenza virus, meningitis virus, measles virus, mumps virus, rubella, pertussis, encephalitis virus, papilloma virus, yellow fever virus, respiratory syncytial virus, parvovirus, chikungunya virus, haemorrhagic fever viruses, Klebsiella, and Herpes viruses, particularly, varicella, cytomegalovirus and Epstein-Barr virus.

The scIL-12 polypeptide and polynucleotide compositions of the invention are also useful for enhancing the immune response to bacterial vaccines, such as, but not limited to, vaccines against leprosy, tuberculosis, Yersinia pestis, Typhoid fever, pneumococcal bacteria, tetanus and anthrax, among others.

Similarly, polypeptides and polynucleotides of the invention are also useful for enhancing the immune response to vaccines against parasitic infections (such as leishmaniasis and malaria, among others) and vaccines against protozoan infections (e.g., T. cruzii) or helminths, e.g., Schistosoma.

The scIL-12 polypeptide and polynucleotide compositions of the invention are also useful for enhancing the immune response to a therapeutic cancer vaccine. A cancer vaccine may comprise an antigen expressed on the surface of a cancer cell. This antigen may be naturally present on the cancer cell. Alternatively, the cancer cell may be manipulated ex vivo and transfected with a selected antigen, which it then expresses when introduced into the patient. A nonlimiting example of a cancer vaccine which may be enhanced by polynucleotides and polypeptides of the invention includes Sipuleucel-T (Provenge®).

Methods of formulating and administering vaccine adjuvants are known in the art, such as the methods described in U.S. Pat. No. 5,571,515, which are herein incorporated by reference.

### Methods of Treating Cancer

The scIL-12 polypeptide and polynucleotide compositions according to the present invention can be used to treat a cancer. Non-limiting examples of cancers that can be treated according to the invention include without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, mesothelioma, osteogenic sarcoma, primary macroglobulinemia, and retinoblastoma, and the like.

The invention provides a method of treating cancer comprising administering a scIL-12 polyptide of the invention to a patient in a therapeutically effective amount. In certain embodiments the scIL-12 polypeptide is administered intratumorally.

The invention also provide a method of treating cancer comprising administering a scIL-12 polynucleotide of the invention to a patient in an amount sufficient to produce a therapeutically effective dose of scIL-12 polypeptide. In certain embodiments the scIL-12 polypeptide is administered intratumorally. In additional embodiments, the scIL-12 polynucleotide is contained in an expression vector. In a preferred embodiment, the expression vector is an adenoviral vector or adeno-associated viral (AAV) vector.

The scIL-12 polynucleotides and polypeptides of the invention may be administered in combination with one or more therapeutic agents and/or procedures in the treatment, prevention, amelioration and/or cure of cancers.

In a specific embodiment, scIL-12 polynucleotides and polypeptides of the invention are administered in combination with one or more chemotherapeutic useful in the treatment of cancers including, but not limited to Alkylating agents; Nitrogen mustards (mechlorethamine, cyclophosphamide, ifosfamide, melphalan, chlorambucil); Nitrosoureas (carmustine (BCNU), lomustine (CCNU), semustine (methyl-CCNU), Ethylenimine/Methyl-melamine, thriethylenemelamine (TEM), triethylene thiophosphoramide (thiotepa), hexamethylmelamine (HMM, altretamine)); Alkyl sulfonates (busulfan); Triazines (dacarbazine (DTIC)); Folic Acid analogs (methotrexate, Trimetrexate, Pemetrexed); Pyrimidine analogs (5-fluorouracil fluorodeoxyuridine, gemcitabine, cytosine arabinoside (AraC, cytarabine), 5-azacytidine, 2,2′-difluorodeoxy-cytidine); Purine analogs (6-mercaptopurine, 6-thioguanine, azathioprine, 2′-deoxycoformycin (pentostatin), erythrohydroxynonyl-adenine (EHNA), fludarabine phosphate, 2-chlorodeoxyadenosine (cladribine, 2-CdA)); Type I Topoisomerase Inhibitors (camptothecin, topotecan, irinotecan); Biological response modifiers (IL-2, G-CSF, GM-CSF); Differentiation Agents (retinoic acid derivatives, Hormones and antagonists); Adrenocorticosteroids/antagonists (prednisone and equivalents, dexamethasone, ainoglutethimide); Progestins (hydroxyprogesterone caproate, medroxyprogesterone acetate, megestrol acetate); Estrogens (diethylstilbestrol, ethynyl estradiol/equivalents); Antiestrogen (tamoxifen); Androgens (testosterone propionate, fluoxymesterone/equivalents); Antiandrogens (flutamide, gonadotropin-releasing hormone analogs, leuprolide); Nonsteroidal antiandrogens (flutamide); Natural products; Antimitotic drugs; Taxanes (paclitaxel, Vinca alkaloids, vinblastine (VLB), vincristine, vinorelbine, Taxotere (docetaxel), estramustine, estramustine phosphate); Epipodophylotoxins (etoposide, teniposide); Antibiotics (actimomycin D, daunomycin (rubido-mycin), doxorubicin (adria-mycin), mitoxantroneidarubicin, bleomycin, splicamycin (mithramycin), mitomycinC, dactinomycin, aphidicolin); Enzymes (L-asparaginase, L-arginase); Radiosensitizers (metronidazole, misonidazole, desmethylmisonidazole, pimonidazole, etanidazole, nimorazole, RSU 1069, EO9, RB 6145, SR4233, nicotinamide, 5 -bromodcozyuridine, 5 -iododcoxyuridine, bromodeoxycytidine); Platinium coordination complexes (cisplatin, Carboplatin, oxaliplatin, Anthracenedione, mitoxantrone); Substituted urea (hydroxyurea); Oxazaphosphorines (cyclophosphamide; ifosfamide; trofosfamide; mafosfamide (NSC 345842), glufosfamide (D19575, beta-D-glucosylisophosphoramide mustard), S-(−)-bromofosfamide (CBM-11), NSC 612567 (aldophosphamide perhydrothiazine); NSC 613060 (aldophosphamide thiazolidine); isophosphoramide mustard; palifosfamide lysine); Methylhydrazine derivatives (N-methylhydrazine (MIH), procarbazine); Adrenocortical suppressant (mitotane (o,p′-DDD), ainoglutethimide); Cytokines (interferon (alpha, beta, gamma), interleukin-2); Photosensitizers (hematoporphyrin derivatives, Photofrin, benzoporphyrin derivatives, Npe6, tin etioporphyrin (SnET2), pheoboride-a, bacteriochlorophyll-a, naphthalocyanines, phthalocyanines, zinc phthalocyanines); and Radiation (X-ray, ultraviolet light, gamma radiation, visible light, infrared radiation, microwave radiation).

### Modes of Administration

The scIL-12 polypeptides and polynucleotides may be administered to the subject systemically or locally (e.g., at the site of the disease or disorder). Systemic administration may be by any suitable method, including subcutaneously and intravenously. Local administration may be by any suitable method, including without limitation, intraperitoneally, intrathecally, intraventricularly, or by direct injection into a tissue or organ, such as intratumoral injection.

In certain embodiments, scIL-12 polynucleotide expression is controlled by a ligand-inducible gene switch system, such as described, for example, in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/U52002/005706 (WO 2002/066614); U.S. Pat. Nos. 7,531,326; 8,236,556; 8,598,409; PCT/US2002/005090 (WO 2002/066612); U.S. application Ser. No. 10/468,193 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); and, U.S. application Ser. No. 12/241,018 (U.S. Pub. No. 20090136465). In these embodiments, once the scIL-12 polynucleotides under the control of a gene switch have been introduced to the subject, an activating ligand may be administered to induce expression of the scIL-12 polypeptide of the invention. The ligand may be administered by any suitable method, either systemically (e.g., orally, intravenously) or locally (e.g., intraperitoneally, intrathecally, intraventricularly, direct injection into the tissue or organ where the disease or disorder is occurring, including intratumorally). The optimal timing of ligand administration can be determined for each type of cell and disease or disorder using only routine techniques.

In certain embodiments, scIL-12 polynucleotides are introduced into in vitro engineered cells such as immune cells (e.g., dendritic cells, T cells, Natural Killer cells) or stem cells (e.g., mesenchymal stem cells, endometrial stem cells, endometrial regenerative cell (ERC), embryonic stem cells), which conditionally express a scIL-12 polypeptide under the control of a gene switch, which can be activated by an activating ligand. Such methods are described in detail, for example, in: PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. application Ser. No. 14/001,943 (U.S. Pub. No. [Pending]).

In one embodiment, immune cells or stem cells are transfected with an adenovirus vector or an adeno-associated virus vector comprising a scIL-12 polynucleotide to produce in vitro engineered cells.

In one embodiment the in vitro engineered immune cells or stem cells are autologous cells. In another embodiment the in vitro engineered immune cells or stem cells are allogeneic.

One embodiment of the invention provides a method for treating a tumor, comprising the steps in order of: 1) administering intratumorally in a mammal a population of in vitro engineered immune cells or stem cells containing a scIL-12 vector under the control of a gene switch; and 2) administering to said mammal a therapeutically effective amount of an activating ligand.

In certain embodiments the mammal is a human. In other embodiments the mammal is a dog, a cat, or a horse.

In one embodiment, the activating ligand is administered at substantially the same time as the composition comprising the in vitro engineered cells or the vector, e.g., adenoviral or adeno-associated viral vector, e.g., within one hour before or after administration of the cells or the vector compositions. In another embodiment, the activating ligand is administered at or less than about 24 hours after administration of the in vitro engineered immune cells or stem cells, or the vector. In still another embodiment, the activating ligand is administered at or less than about 48 hours after the in vitro engineered immune cells or stem cells, or the vector. In another embodiment, the ligand is RG-115932. In another embodiment, the ligand is administered at a dose of about 1 to 50 mg/kg/day. In another embodiment, the ligand is administered at a dose of about 30 mg/kg/day. In another embodiment, the ligand is administered daily for a period of 7 to 28 days. In another embodiment, the ligand is administered daily for a period of 14 days. In another embodiment, about 1×10⁶ to 1×10⁸ cells are administered. In another embodiment, about 1×10⁷ cells are administered.

Having provided for the substantially pure polypeptides, biologically active fragments thereof and recombinant polynucleotides encoding them, the present invention also provides cells comprising each of them. By appropriate introduction techniques well known in the field, cells comprising them may be produced. See, e.g., Sambrook et al. (1989).

### Host Cells and Non-human Organisms

Another aspect of the present invention involves cells comprising an isolated polynucleotide encoding a scIL-12 polypeptide of the present invention. In a specific embodiment, the invention relates to an isolated host cell comprising a vector comprising a polynucleotide encoding a scIL-12 polypeptide of the present invention. The present invention also relates to an isolated host cell comprising an expression vector according to the invention. In another specific embodiment, the invention relates to an isolated host cell comprising a gene expression cassette comprising a polynucleotide encoding a scIL-12 polypeptide of the present invention. In another specific embodiment, the invention relates to an isolated host cell transfected with a g ene expression modulation system comprising a polynucleotide encoding a scIL-12 polypeptide of the present invention. In still another embodiment, the invention relates to a method for producing a scIL-12 polypeptide, wherein the method comprises culturing an isolated host cell comprising a polynucleotide encoding a scIL-12 polypeptide of the present invention in culture medium under conditions permitting expression of the polynucleotide encoding the scIL-12 polypeptide, and isolating the scIL-12 polypeptide from the culture.

In one embodiment, the isolated host cell is a prokaryotic host cell or a eukaryotic host cell. In another specific embodiment, the isolated host cell is an invertebrate host cell or a vertebrate host cell. Preferably, the isolated host cell is selected from the group consisting of a bacterial cell, a fungal cell, a yeast cell, a nematode cell, an insect cell, a fish cell, a plant cell, an avian cell, an animal cell, and a mammalian cell. For example but without limitation, the isolated host cell may be a yeast cell, a nematode cell, an insect cell, a plant cell, a zebrafish cell, a chicken cell, a hamster cell, a mouse cell, a rat cell, a rabbit cell, a cat cell, a dog cell, a bovine cell, a goat cell, a cow cell, a pig cell, a horse cell, a sheep cell, or a non-human primate cell (for example, a simian cell, a monkey cell, a chimpanzee cell), or a human cell.

Examples of host cells include, but are not limited to, fungal or yeast species such as Aspergillus, Trichoderma, Saccharomyces, Pichia, Candida, Hansenula, or bacterial species such as those in the genera Synechocystis, Synechococcus, Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella; animal; and mammalian host cells.

In one embodiment, the isolated host cell is a yeast cell selected from the group consisting of a Saccharomyces, a Pichia, and a Candida host cell.

In another embodiment, the isolated host cell is a Caenorhabdus elegans nematode cell.

In another embodiment, the isolated host cell is a mammalian cell selected from the group consisting of a hamster cell, a mouse cell, a rat cell, a rabbit cell, a cat cell, a dog cell, a bovine cell, a goat cell, a cow cell, a pig cell, a horse cell, a sheep cell, a non-human primate cell (such as a monkey cell or a chimpanzee cell), and a human cell.

Host cell transformation is well known in the art and may be achieved by a variety of methods including but not limited to electroporation, viral infection, plasmid/vector transfection, non-viral vector mediated transfection, Agrobacterium-mediated transformation, particle bombardment, and the like. Expression of desired gene products involves culturing the transformed host cells under suitable conditions and inducing expression of the transformed gene. Culture conditions and gene expression protocols in prokaryotic and eukaryotic cells are well known in the art (see General Methods section of Examples). Cells may be harvested and the gene products isolated according to protocols specific for the gene product.

In addition, a host cell may be chosen that modulates the expression of the transfected polynucleotide, or modifies and processes the polypeptide product in a specific fashion desired. Different host cells have characteristic and specific mechanisms for the translational and post-translational processing and modification [e.g., glycosylation, cleavage (e.g., of signal sequence)] of proteins. Appropriate cell lines or host systems can be chosen to ensure the desired modification and processing of the foreign protein expressed. For example, expression in a bacterial system can be used to produce a non-glycosylated core protein product. However, a polypeptide expressed in bacteria may not be properly folded. Expression in yeast can produce a glycosylated product. Expression in eukaryotic cells can increase the likelihood of “native” glycosylation and folding of a heterologous protein. Moreover, expression in mammalian cells can provide a tool for reconstituting, or constituting, the polypeptide's activity. Furthermore, different vector/host expression systems may affect processing reactions, such as proteolytic cleavages, to a different extent.

Applicants' invention also relates to a non-human organism comprising an isolated host cell according to the invention. In a specific embodiment, the non-human organism is a prokaryotic organism or a eukaryotic organism. In another specific embodiment, the non-human organism is an invertebrate organism or a vertebrate organism.

In certain embodiments, the non-human organism is selected from the group consisting of a bacterium, a fungus, a yeast, a nematode, an insect, a fish, a plant, a bird, an animal, and a mammal. More preferably, the non-human organism is a yeast, a nematode, an insect, a plant, a zebrafish, a chicken, a hamster, a mouse, a rat, a rabbit, a cat, a dog, a bovine, a goat, a cow, a pig, a horse, a sheep, or a non-human primate (such as a simian, a monkey, or a chimpanzee).

The present invention may be better understood by reference to the following non-limiting Examples, which are provided as exemplary of the invention.

### EXAMPLES

### General Molecular Biology Techniques

In accordance with the present invention there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York (herein “Green & Sambrook, 2012”); DNA Cloning: A Practical Approach, Volumes I and II, Second Edition (D. M. Glover and B. D. Hames, eds. 1995); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Culture of Animal Cells: A Manual of Basic Technique and Specialized Applications [R. I. Freshney (2010)]; Immobilized Cells And Enzymes [IRL Press, (1986)]; B. Perbal, A Practical Guide To Molecular Cloning, Second Edition (1988); F. M. Ausubel et al. (eds.), Current Protocols in Molecular Biology, John Wiley & Sons, Inc. (2013).

Conventional cloning vehicles include pBR322 and pUC type plasmids and phages of the M13 series. These may be obtained commercially (e.g., Life Technologies Corporation; Promega Corporation).

For ligation, DNA fragments may be separated according to their size by agarose or acrylamide gel electrophoresis, extracted with phenol or with a phenol/chloroform mixture, precipitated with ethanol and then incubated in the presence of phage T4 DNA ligase (New England Biolabs, Inc.) according to the supplier's recommendations.

The filling in of 5′ protruding ends may be performed with the Klenow fragment of E. coli DNA polymerase I (New England Biolabs, Inc.) according to the supplier's specifications. The destruction of 3′ protruding ends is performed in the presence of phage T4 DNA polymerase (New England Biolabs, Inc.) used according to the manufacturer's recommendations. The destruction of 5′ protruding ends is performed by a controlled treatment with S1 nuclease.

Mutagenesis directed in vitro by synthetic oligodeoxynucleotides may be performed according to the method developed by Taylor et al. [Nucleic Acids Res. 13 (1985) 8749-8764] using commercial kits such as those distributed by Life Technologies Corp. and Agilent Technologies, Inc.

The enzymatic amplification of DNA fragments by PCR [Polymerase-catalyzed Chain Reaction, Saiki R. K. et al., Science 230 (1985) 1350-1354; Mullis K. B. and Faloona F. A., Meth. Enzym. 155 (1987) 335-350] technique may be performed using a “DNA thermal cycler” (Life Technologies Corp.) according to the manufacturer's specifications.

Verification of nucleotide sequences may be performed by the method developed by Sanger et al. [Proc. Natl. Acad. Sci. USA, 74 (1977) 5463-5467] using commercial kits such as those distributed by GE Healthcare and Life Technologies Corp.

Plasmid DNAs may be purified by the Qiagen Plasmid Purification System according to the manufacture's instruction.

### Example 1

### Design of scIL-12 Fusion Proteins

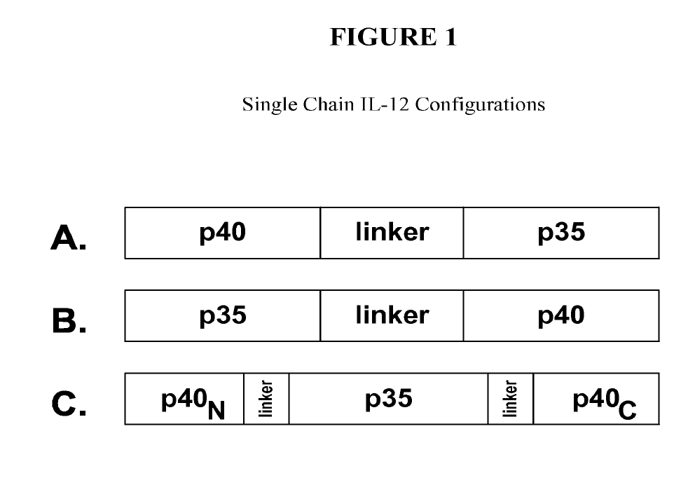

Single chain IL-12 molecules were designed to have one of two configurations, illustrated in FIG. 1:
1) The p40-linker-p35 configuration (FIG. 1A) contains the full-length p40 subunit (including wild type signal peptide) fused to the mature p35 subunit (without signal peptide) via a peptide linker;
2) The p35-linker-p40 configuration (FIG. 1B) contains the full-length p35 subunit (including wild type signal peptide) fused to the mature p40 subunit (without signal peptide) via a peptide linker; and
3) The p40N-p35-p40C insert configuration (FIG. 1C) comprising, from N- to C-terminus:
   (i) a first IL-12 p40 domain (p40N),
   (ii) an optional first peptide linker,
   (iii) an IL-12 p35 domain,
   (iv) an optional second peptide linker, and
   (v) a second IL-12 p40 domain (p40C).

Specific human scIL-12 constructs are summarized in Table 1. Amino acid residues specified by number in the Description column refer to the amino acid numbering of the full-length human p40 or p35 subunits shown in SEQ ID NOs: 2 and 4, respectively. For example, the nucleic acid and amino acid sequences of scIL-12 Construct ID 1481273, corresponding to SEQ ID NOs: 9 and 10, respectively, is a p40N-p35-p40C insert configuration; and was designed to contain, from N- to C-terminus, a first p40 domain (p40N) consisting of amino acids 1 to 293 of SEQ ID NO: 2, a first linker sequence of TPS (Thr-Pro-Ser; SEQ ID NO: 41), a mature p35 sequence consisting of amino acids 57 to 253 of SEQ ID NO: 4, a second peptide linker sequence of GPAPTS (Gly-Pro-Ala-Pro-Thr-Ser; SEQ ID NO: 42), and a second p40 domain (p40C) consisting of amino acids 294 to 328 of SEQ ID NO: 2.

Construct ID 1481272 (SEQ ID NOs: 11 and 12) is also a p40N-p35-40C insert configuration, but the p35 insert occurs between amino acid residues 259 and 260 of the p40 subunit.

The remaining scIL-12 designs (Construct IDs 1480533 to 1480546) represent p40-p35 or p35-p40 single chain IL-12 molecules with various linkers as indicated in Table 1.

Parallel mouse constructs were also designed, using the mouse p40 and p35 sequences (SEQ ID NOs: 5-8) instead of human IL-12 sequences.

TABLE 1
Human scIL-12 constructs

| | DNA | Protein | |
|---|---|---|---|
| Construct | SEQ | SEQ | |
| ID | ID NO | ID NO | Description |
| 1481273 | 9 | 10 | p40N(₁₋₂₉₃₎-TPS-p35₍₅₇₋₂₅₃₎-GPAPTS-p40C₍₂₉₄₋₃₂₈₎ |
| 1481272 | 11 | 12 | p40N₍₁₋₂₅₉₎-GS-p35₍₅₇₋₂₅₃₎-PQTPGP-p40C₍₂₆₀₋₃₂₈₎ |
| 1480533 | 13 | 14 | p40₍₁₋₃₂₈₎-RSPVSGDNAFPAPTG-p35₍₅₇₋₂₅₃₎ |
| 1480534 | 15 | 16 | p40₍₁₋₃₂₈₎-RSQPVPTRDLEVPLTG-p35₍₅₇₋₂₅₃₎ |
| 1480535 | 17 | 18 | p40₍₁₋₃₂₈₎-RSGTPPQTGLEKPTGTG-P35₍₅₇₋₂₅₃₎ |
| 1480536 | 19 | 20 | p40₍₁₋₃₂₈₎-SDVTGNTGNATYTIT-p35₍₅₇₋₂₅₃₎ |
| 1480537 | 21 | 22 | p40₍₁₋₃₂₈₎-GSPKDGPEIPPTGGT-p35₍₅₇₋₂₅₃₎ |
| 1480538 | 23 | 24 | p40₍₁₋₃₂₈₎-GRNAPGSPPTGNYKLEP-p35₍₅₇₋₂₅₃₎ |
| 1480539 | 25 | 26 | p40₍₁₋₃₂₈₎-QKGSVGFTDPEVHQSTNL-P35₍₅₇₋₂₅₃₎ |
| 1480540 | 27 | 28 | p40₍₁₋₃₂₈₎-GNVPELPDTTEHSRT-p35₍₅₇₋₂₅₃₎ |
| 1480541 | 29 | 30 | p40₍₁₋₃₂₈₎-GRSHPVQPYPGAFVKEPIP-p35₍₅₇₋₂₅₃₎ |
| 1480542 | 31 | 32 | P40₍₁₋₃₂₈₎-PERKERISEQTYQLS-p35₍₅₇₋₂₅₃₎ |
| 1480543 | 33 | 34 | P40₍₁₋₃₂₈₎-(G₄S)₃-P35₍₅₇₋₂₅₃₎ |
| 1480544 | 35 | 36 | P40₍₁₋₃₂₈₎-G₆S-P35₍₅₇₋₂₅₃₎ |
| 1480545 | 37 | 38 | p35₍₃₅₋₂₅₃₎-RSDVNSRTGPSGATPPSGNPYTITG-p40₍₂₃₋₃₂₈₎ |
| 1480546 | 39 | 40 | P35₍₃₅₋₂₅₃₎-PAPTPSNGSPKDGPEIPPTGG-p40₍₂₃₋₃₂₈₎ |

Embodiments of the invention include, without limitation, the scIL-12 constructs indicated in Table 1 above. The scIL-12 constructs of the invention may comprise, or may not comprise, a signal peptide sequence (whether synthesized with or without a signal peptide or as may occur as a result of polylpeptide cleavage in the secreted form subsequent to in vitro or in vivo expression and post-translational processing). For example, but without limitation, with respect to scIL-12 Construct No. 1481273 (p40N₍₁₋₂₉₃₎-TPS-p35₍₅₇₋₂₅₃₎-GPAPTS-p40C₍₂₉₄₋₃₂₈₎ embodiments of the invention also include this polypeptide sequence without a signal peptide (e.g., p40N₍₂₃₋₂₉₃₎-TPS-p35₍₅₇₋₂₅₃₎-GPAPTS-p40C₍₂₉₄₋₃₂₈₎. Likewise, without limitation, embodiments of the invention include any of the remaining scIL-12 constructs shown in Table 1 without a signal peptide.

### Example 2

### Expression of scIL-12 Fusion Proteins in CHO Cells

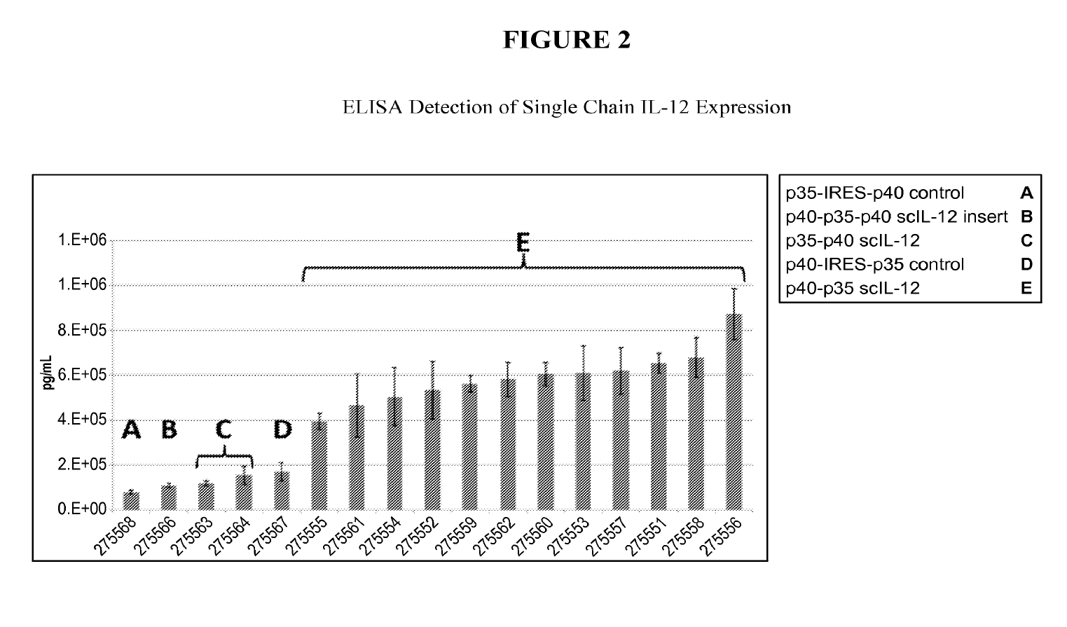

Vectors were constructed containing either human or murine scIL-12 (in all cases cloned between NheI and ClaI sites) along with a 5′UTR element derived from human GAPDH, a synthetic 3′UTR element and with transgene expression under control of a constitutive CMV promoter. Vectors encoding human or mouse scIL-12 constructs were transiently transfected into CHO-K1 cells (ATCC Accession CCL-61) in triplicate using standard high-throughput transfection methods. Briefly, CHO-K1 cells were trypsinized, counted and re-suspended at 120,000 cells/ml in whole growth media (F12-Ham (Sigma)+L-Glutamine (Gibco)+10% FBS (Atlanta Biologicals). One-hundred fifty (150) micro liters of the cell suspension was added to a 96-well cell culture plate (Corning). Plasmid DNA was prepared at 100 ng/μl in sterile water and complexed with Fugene 6 reagent (Promega) at a 3:1 DNA to Fugene 6 ratio. Five (5) micro liters of the DNA/Fugene6 complex was added to the 96-well plate containing the cells. The cells were then incubated at 37° C. for 48 hours. Following incubation the culture supernatant was harvested, and frozen at −80° C. until used for ELISA assays. Positive controls included vectors expressing two-chain IL-12 (p35-IRES-p40 and p40-IRES-p35, labeled in FIG. 2 as bars A and D, respectively). Culture supernatants from transfected CHO-K1 cells were diluted 1:10, 1:100, and 1:1000 in R&D Systems Reagent Diluent+10% conditioned CHO-K1 media.

Expression of scIL-12 was detected by ELISA assays run according to the manufacturer's instructions. R&D Systems, catalog #DY419 (mouse IL-12 ELISA) and #DY1270 (human IL-12 ELISA). Nine samples per vector were analyzed.

Human scIL-12 expression was detected in 20 of the 36 vectors evaluated, and ranged from 500 pg/mL to 900 ng/mL. See FIG. 2. Mouse scIL-12 expression was detected in 18 of the 36 vectors tested. Mouse scIL-12 expression ranged from 385 pg/mL to 1.8 μg/mL (data not shown). For both human and mouse constructs, the p40-linker-p35 configuration demonstrated higher expression levels than the p35-linker-p40 configuration and two-chain (bicistronic) IL-12, suggesting that scIL-12 with p40-linker-p35 topology has enhanced expression, folding and/or heterodimeric assembly as compared to the p35-linker-p40 single chain configuration and two-chain IL-12.

Surprisingly, the human scIL-12 construct ID 1481273, having the configuration:
p40N_{(1 to 293)}-TPS-p35₍₅₇₋₂₅₃₎-GPAPTS-p40C_{(294 to 328)} resulted in scIL-12 protein expression that was similar to levels produced by two-chain (bicistronic) vectors (p40-IRES-p35 and p35-IRES-p40) and single chain p35-linker-p40 configuration, although not as high as the p40-linker-p35 configuration. See FIG. 2. Similar expression patterns were observed for the mouse scIL-12 designs. Construct ID 1481272, having the configuration p40N₍₁₋₂₅₉₎-GS-p35₍₅₇₋₂₅₃₎-PQTPGP-p40C₍₂₆₀₋₃₂₈₎, was found not to express detectable protein.

### Example 3

### scIL-12 Stimulation of IFN-gamma Production in NK Cells

Natural Killer (NK) cells secrete interferon gamma (IFN-gamma) in response to IL-12 exposure. Therefore, we measured IFN-gamma production in NK-92 cells (ATCC Accession CRL-2407), a human Natural Killer cell line, in a bioassay to detect the functional activity of scIL-12 designs of the invention.

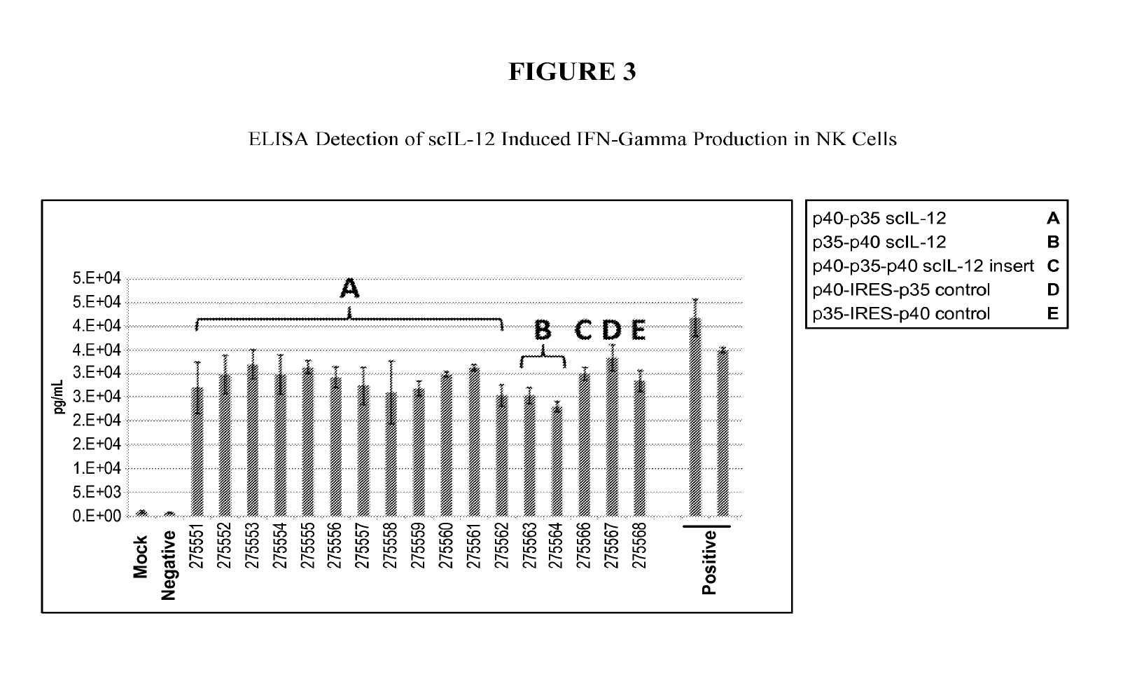

NK-92 cells were cultured according to the manufacturer's instructions using the recommended culture medium (Alpha Minimum Essential medium without ribonucleosides and deoxyribonucleosides, with 2 mM L-glutamine; 1.5 g/L sodium bicarbonate; 0.2 mM inositol; 0.1 mM 2-mercaptoethanol; 0.02 mM folic acid; 100-200 U/ml recombinant IL-2; adjusted to a final concentration of 12.5% horse serum and 12.5% fetal bovine serum). The NK-92 cells were sub-cultured 24-48 hours prior to use in the assay. On the day of the assay, the NK-92 cells were counted by staining with Trypan Blue and seeded into 96-well plates at 5×10⁴ cells per well. CHO-K1/scIL-12 culture supernatants obtained in Example 2 were diluted 1:5 in NK-92 whole growth media and added to the NK-92 cells. Controls included culture supernatants from un-transfected CHO-K1 cells (labeled “Mock” in FIG. 3) and from CHO-K1 cells transfected with plasmid not expressing IL-12 (i.e., CMV-GFP; labeled “Negative” in FIG. 3) as negative controls; and a positive control consisting of commercially available recombinant human IL-12 (R&D Systems), which was tested at 1250 ng /ml or 125 ng /ml (left and right positive controls bars, respectively, in FIG. 3). NK-92 cell culture supernatants were harvested after 48 hours, and diluted 1:10, 1:100, and 1:1000 in R&D Systems Reagent Diluent. The amount of IFN-gamma in the culture medium was determined using the R&D Systems Human IFN-gamma Duoset ELISA kit (Catalog #DY285). Nine samples per vector were analyzed.

Human scIL-12 proteins stimulated human IFN-gamma production in NK-92. Human IFN-gamma expression ranged from 600 pg/mL to 33 ng /mL. See FIG. 3. Similar IFN-gamma levels were observed for the mouse scIL-12 constructs.

Surprisingly, scIL-12 Construct ID 1481273, which exhibited relatively low protein expression levels (see Example 2), demonstrated equivalent activity to recombinant two-chain IL-12 and to p40-p35 single chain constructs in the NK-92 bioassay, suggesting that Construct ID 1481273 may be more active on a per-molecule basis.

### Example 4

### Exemplary IL-12 Functional Assay using NK Cells

The assay described herein may be used to measure the ability of IL-12 polypeptides (e.g., recombinantly produced heterologous p35/p40 (p70) polypeptides and single chain IL-12 (p70) polypeptides) to induce interferon-gamma (“IFN-gamma” or “IFN-g”) production in immune cells (such as, but not limited to, NK-92 cells) in a dose-dependent manner. It is understood that those skilled in the field of the invention may modify assays and procedures, as well as used different assays, to measure biological activity of IL-12.

Natural Killer (NK) cells secrete interferon gamma (IFN-gamma) in response to contact with (exposure to) IL-12. Accordingly, in this assay, NK-92 cells are stimulated with escalating doses of recombinant human and/or mouse IL-12 for 24 hours. Subsequently, IFN-gamma in the NK-92 supernatant is measured by ELISA. As a result, IFN-gamma expression decreases as IL-12 dose decreases (or conversely, up to a certain level of dose saturation, IFN-gamma expression increases as IL-12 doses increase).

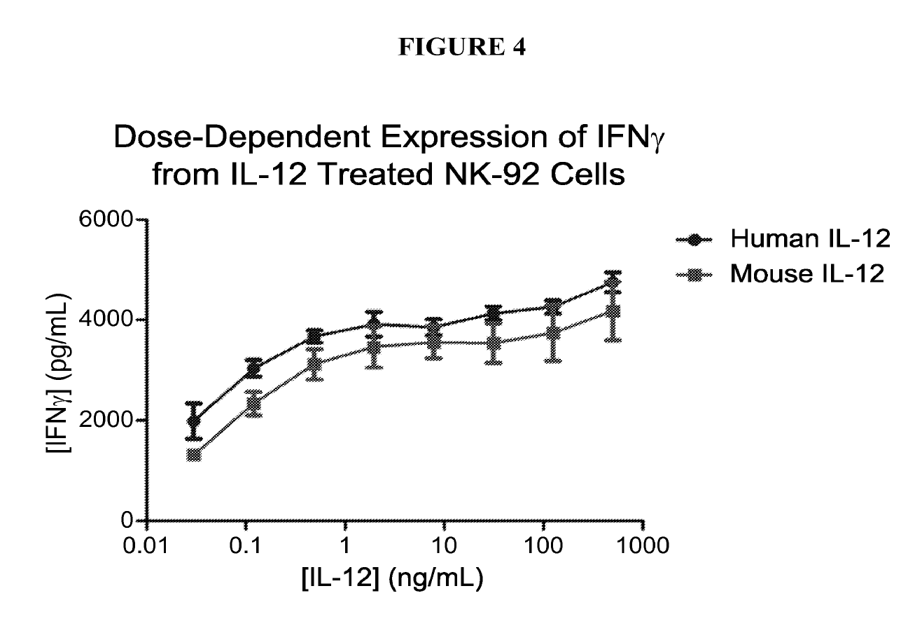

FIG. 4 shows a typical result obtained in a dose-response graph (or “curve”) using human IL-12 and mouse IL-12 where dose-dependent expression of IFN-gamma by NK-92 cells treated with escalating doses of IL-12 for 24 hours was measured. In this assay, NK-92 cells were seeded at 50,000 cells/well and treated with 0.06-1000 nanograms (ng)/mL recombinant human or mouse IL-12. NK-92 supernatants were harvested 24 hours later and tested by ELISA detection of human IFN-gamma. Data depicted shows average IFN-gamma expression from 3 replicate samples, calculated based on the 1:5 sample dilution. Error bars show standard deviation. The ELISA was run in the presence of 20% NK-92 conditioned media to account for endogenous IFN-gamma expression from cells. The results demonstrate that IFN-gamma expression from NK-92 cells is IL-12 dose-dependent. Notably, NK-92 cells responded similarly to both human and mouse IL-12.

The protocol used in this assay utilized NK-92 cells harvested and centrifuged at 1200 rpm for 5 minutes. Spent media was removed and replaced with 1/5 volume of fresh medium. Cells were counted using a hemacytometer and resuspended at 1×106 cells/mL. Fifty microliters per well of NK-92 cells were plated into 96 well tissue-culture treated plates and incubated at 37° C. with 5% CO2 incubator until ready to dose. A dilution curve of rhIL-12 (recombinant human IL-12) or rmIL-12 (recombinant mouse IL-12) was prepared by diluting IL-12 in NK-92 culture media at final concentrations of 1000, 250, 62.5, 15.63, 3.91, 0.98, 0.24 and 0.06 nanograms/mL of IL-12. Each well of a 96-well plate (with NK92 cells) was dosed with 50 microliters per well of IL-12; plates were then incubated for 24 hours.

NK92 cell plates were subsequently centrifuged and cell culture supernatants were harvested and stored at 4 degrees C. until ready to assay (for IFN-gamma). For the Interferon-gamma ELISA, a standard curve of recombinant protein was prepared at concentrations of 1000, 500, 250, 125, 62.5, 31.3, 15.6 and 0 picograms (pg)/mL of IFN-gamma. ELISA analysis was performed using standard procedures; comparing IFN-gamma standards to 1:5, 1:25, 1:125, 1:625 and 1:3125 dilutions of NK92 cell supernatants. Results obtained are shown in FIG. 4.

### Example 5

### Production and Biological Activity Testing of Single Chain IL-12 Constructs

Experiments were performed to express single chain IL-12 constructs in 293T cells and measure IL-12 dose-response biological activity (i.e., ability of IL-12 polypeptides to stimulate IFN-gamma production from NK-92 cells in a dose-dependent manner). These experiments further show that single chain IL-12 (scIL-12) polypeptides of the invention (wherein the length of linker sequences, if any, is minimized by inserting IL-12 p35 polypeptide sequences within an IL-12 p40 polypeptide) retains dose-dependent IL-12 biological activity similar to that of native IL-12.

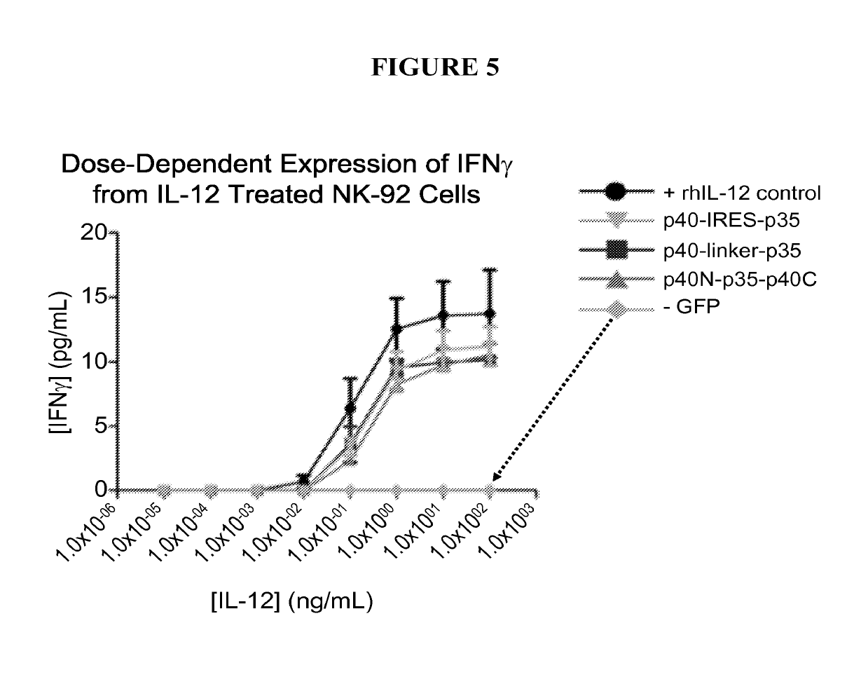

Three IL-12 constructs were expressed by transient transfection of 293T cells for 72 hours. Transfected supernatants were harvested and IL-12 p70 (i.e., p35/p40 heterodimers or single chain IL-12 polypeptides) was quantitated by ELISA. IL-12 constructs were then tested in a functional assay by treating NK-92 cells with escalating doses of IL-12 (0.00001-100 nanograms/mL). Recombinant human IL-12 (previously demonstrated to induce dose-dependent expression of IFN-gamma from NK-92 cells) was included as a positive control. 293T cell supernatants from cells transfected with a GFP control vector was included as a negative control. Results show that both single chain and native IL-12 proteins induced dose-dependent IFN-gamma expression by NK-92 cells. Furthermore, the level of induction was similar across each of the three IL-12 constructs as well as the positive control. No IFN-gamma expression was observed from NK-92 cells treated with 293T GFP-transfected supernatants. See, FIG. 5.

TABLE 2
IL-12 Constructs For Induction of Interferon-Gamma by NK92 Cells

| IL-12 Construct | | |
|---|---|---|
| (SEQ ID Nos) | IL-12 Linker | DNA Vector |
| p40-linker-p35 | -GGGGGGS- | 275562 |
| (SEQ ID NOs: 35 & 36) | | |
| p40N-p35-p40C | -TPS-p35-GPAPTS- | 275566 |
| (SEQ ID NOs: 9 & 10) | | |
| p40/p35 | None | 275567 |
| (SEQ ID NOs: 1/3 & 2/4) | (Heterodimeric p35/p40 wit | |
| | IRES separating p35 and | |
| | p40 open reading frames) | |
| CMV-GFP | N/A | 40022 |

### IFN-gamma Quantitation Procedure

Cell Seeding: One day prior to transfection, 293T cells were seeded in a 6 well dish at 7.58e5 cells/well (media composition of 10% FBS, DMEM, 1× GLUTAMAX™ (Life Technologies Inc.)) and incubated overnight at 37 degrees C. in air supplemented with 5% carbon dioxide.

Transfection: Next day, DNA vectors were diluted to a final concentration of 100 micrograms/mL DNA (starting DNA concentrations ranged from 1000 to ˜1300 micrograms/mL) . Transfection mixes were prepared with 22 microliters FUGENE® 6 transfection reagent (Promega Corp., Madison, Wis., USA), 308 microliters OPTI-MEM® cell culture media (Life Technologies Inc., Grand Island, N.Y., USA), and 36.7 microliters DNA solution in a 15 mL conical polystyrene tube. Tube was agitated quickly but gently to mix and incubated at 15 minutes at room temperature. 167 microliters of transfection mixture was added to each well in 6-well dishes with vectors 275566 and 275567 in duplicate wells. Plates were incubated at 37 degrees C. with 5% carbon dioxide. Seventy-two hours post transfection, cell culture supernatants were harvested and sterile filtered using a 0.2 micron filter and syringe. One-hundred and fifty microliters per sample was used for IL-12 ELISA quantitation. The remainder was stored at -80 degrees C. until used in IFN-gamma assay.

IL-12 ELISA: Commercially available IL-12 ELISA kits (e.g., Human IL-12 p40 (and p70) DUOSET® ELISA from R&D Systems Inc., Minneapolis, Minn., USA) were used according to manufacturer's directions for quantitation of IL-12 in cell culture supernatants. Optical absorbance of ELISA plates at 450 nm were measured. Cell culture supernatants were determined to have the following concentrations of IL-12: p40N-p35-p40C (vector 275566) at 8364 ng/mL; p40/p35 heterodimer (vector 275567) at 28903 ng/mL; and, p40-linker-p35 (vector 275562) at 57197 ng/mL. IL-12 cell culture supernatants were diluted to a final concentration of 2000 ng/mL IL-12. (293T cell GFP-transfected supernatants were diluted with same dilution factor as p40N-p35-p40C supernatants).

NK-92 Functional Assay: On day 1, NK-92 cells were harvested and centrifuged at 1200 rpm for 5 minutes. Spent media was removed and replaced with ⅕ volume of fresh medium. Cells were counted using a hemacytometer. An 88% viable cell count at 2.03e6 c/mL was observed. Eight mL of NK-92 cells at 1e6 cells/mL was prepared (using 4.5 mL cells plus 3.5 mL media to generate 8 mL at 1e6 c/mL). NK92 cells were seeded at 50 microliters per well into two 96 well tissue-culture treated plates and incubated at 37° C/5% CO2 incubator until ready to dose with IL-12. Ten-fold dilutions of IL-12 were prepared to final concentrations of 200, 20, 2, 0.2, 0.02, 0.002, 0.0002 and 0.00002 ng/mL. N K-92 cells in 96-well plates were then dosed (in triplicate or quadruplicate at each concentration) with 50 microliters of IL-12 and returned to incubator for 24 hours. On day 2, the contents of each well in the 96-well plates was transferred to 96-well V-bottom plates and centrifuged at 1200 rpm for 10 minutes. Supernatants were collected (cells were discarded) and stored at 4 degrees C. until used to assay for IFN-gamma quantities. ELISA analysis was performed to quantitate IFN-gamma production according to manufacturer's instructions using a commercially available kit (Human IFN-gamma DUOSET® ELISA from R&D Systems, Inc.) compared to a standard curve of recombinant IFN-gamma. Optical absorbance at 450 nm was measured.

Results: FIG. 5 shows expression of IFN-gamma from NK-92 cells treated with increasing doses of IL-12 (24 hours exposure to IL-12). NK-92 cells were seeded at 50,000 cells/well and treated with 0.00001-100 ng/mL recombinant human IL-12, IL-12 expressed in 293T cells, or 293T supernatant from GFP-transfected cells (negative control). IL-12 induced NK-92 cell supernatants were harvested 24 hours later and tested by human IFN-gamma ELISA. Data show average IFN-gamma expression from 3-4 replicate samples, tested in duplicate at a 1:5 and 1:25 sample dilution (n=12). Error bars show standard deviation. ELISA was run in the presence of 20% NK-92 conditioned media in order to account for endogenous IFN-gamma expression from cells. Data demonstrates that IFN-gamma expression from NK-92 cells is IL-12 dose-dependent for both the transfected samples as well as the recombinant IL-12. IFN-gamma expression appears to be IL-12 specific, as indicated by the lack of IFN-gamma expression from cells treated with the GFP supernatants.

The invention claimed is:

1. A single-chain IL-12 polypeptide comprising, from N- to C-terminus:
i. a first IL-12 p40 domain (p40N),
ii. an optional first peptide linker,
iii. an IL-12 p35 domain,
iv. a optional second peptide linker, and
v. a second IL-12 p40 domain (p40C);
wherein the first IL-12 p40 domain (p40N) is an N-terminal fragment of a p40 subunit; the IL-12 p35 domain is a mature p35 subunit or fragment thereof; and the second IL-12 p40 domain (p40C) is a C-terminal fragment of a p40 subunit.

2. The single chain IL-12 polypeptide of claim 1, which comprises an N-terminal signal peptide domain.

3. The single chain IL-12 polypeptide of claim 1, wherein the polypeptide does not comprise a first peptide linker, does not comprise a second peptide linker, or does not comprise a first peptide linker and does not comprise a second peptide linker.

4. The single chain IL-12 polypeptide of claim 1, wherein the polypeptide does not comprise a first peptide linker, does not comprise a second peptide linker, or does not comprise a first peptide linker and does not comprise a second peptide linker.

5. The single chain IL-12 polypeptide of claim 1, wherein said single-chain IL-12 polypeptide comprises an amino acid sequence at least 80% identical, at least 85% identical, at least 90% identical, at least 95% identical, at least 97% identical, at least 98% identical, at least 99% identical, or 100% identical to the polypeptide sequence of amino acids 23 to 533 or amino acids 1 to 533 of SEQ ID NO: 10.

6. (canceled)

7. (canceled)

8. (canceled)

9. The single chain IL-12 polypeptide of claim 1, wherein the first and second peptide linkers each comprise a number of amino acid residues selected from the group consisting of:
a) 0 amino acids;
b) 1 amino acid;
c) 2 amino acids;
d) 3 amino acids;
e) 4 amino acids;
f) 5 amino acids;
g) 6 amino acids;
h) 7 amino acids;
i) 8 amino acids;
j) 9 amino acids; and,
k) 10 amino acids.

10. The single chain IL-12 polypeptide of claim 9, wherein the amino acid residues in either the first or second peptide linker, or in both peptide linkers, comprise any combination of one or more amino acids selected from the group consisting of:
a) Glycine (Gly);
b) Serine (Ser);
c) Alanine (Ala);
d) Threonine (Thr); and,
e) Proline (Pro).

11. The single chain IL-12 polypeptide of claim 1, wherein the first and second peptide linkers are selected from Thr-Pro-Ser (SEQ ID NO: 41) and Ser-Gly-Pro-Ala-Pro (SEQ ID NO: 42).

12. The single chain IL-12 polypeptide of claim 1, wherein p40N comprises a polypeptide sequence at least 80% identical, at least 85% identical, at least 90% identical, at least 95% identical, at least 97% identical, at least 98% identical, at least 99% identical, or 100% identical to amino acids 18 to 288, amino acids 18 to 298, amino acids 28 to 288, amino acids 28 to 298 or amino acids 1 to 298 of SEQ ID NO: 2.

13. (canceled)

14. The single chain IL-12 polypeptide of claim 1, wherein p40N comprises a fragment of the polypeptide sequence of SEQ ID NO: 2, wherein the first residue of said fragment begins at a position selected from the group consisting of:
a) amino acid residue 18 of SEQ ID NO: 2;
b) amino acid residue 19 of SEQ ID NO: 2;
c) amino acid residue 20 of SEQ ID NO: 2;
d) amino acid residue 21 of SEQ ID NO: 2;
e) amino acid residue 22 of SEQ ID NO: 2;
f) amino acid residue 23 of SEQ ID NO: 2;
g) amino acid residue 24 of SEQ ID NO: 2;
h) amino acid residue 25 of SEQ ID NO: 2;
i) amino acid residue 26 of SEQ ID NO: 2;
j) amino acid residue 27 of SEQ ID NO: 2; and,
k) amino acid residue 28 of SEQ ID NO: 2,
and, wherein the last residue of said fragment ends at a position selected from the group consisting of:
l) amino acid residue 288 of SEQ ID NO: 2;
m) amino acid residue 289 of SEQ ID NO: 2;
n) amino acid residue 290 of SEQ ID NO: 2;
o) amino acid residue 291 of SEQ ID NO: 2;
p) amino acid residue 292 of SEQ ID NO: 2;
q) amino acid residue 293 of SEQ ID NO: 2;
r) amino acid residue 294 of SEQ ID NO: 2;
s) amino acid residue 295 of SEQ ID NO: 2;
t) amino acid residue 296 of SEQ ID NO: 2;
u) amino acid residue 297 of SEQ ID NO: 2; and,
v) amino acid residue 298 of SEQ ID NO: 2.

15. The single chain IL-12 polypeptide of claim 1, wherein p35 comprises a polypeptide sequence at least 80% identical, at least 85% identical, at least 90% identical, at least 95% identical, at least 97% identical, at least 98% identical, at least 99% identical, or 100% identical to amino acids 52 to 247, amino acids 52 to 253, amino acids 62 to 247, amino acids 62 to 253 or amino acids 1 to 253 of SEQ ID NO: 4.

16. (canceled)

17. The single chain IL-12 polypeptide of claim 1, wherein p35 comprises a fragment of the polypeptide sequence of SEQ ID NO:4, wherein the first residue of said fragment begins at a position selected from the group consisting of:
a) amino acid residue 52 of SEQ ID NO: 4;
b) amino acid residue 53 of SEQ ID NO: 4;
c) amino acid residue 54 of SEQ ID NO: 4;
d) amino acid residue 55 of SEQ ID NO: 4;
e) amino acid residue 56 of SEQ ID NO: 4;
f) amino acid residue 57 of SEQ ID NO: 4;
g) amino acid residue 58 of SEQ ID NO: 4;
h) amino acid residue 59 of SEQ ID NO: 4;
i) amino acid residue 60 of SEQ ID NO: 4;
j) amino acid residue 61 of SEQ ID NO: 4; and,
k) amino acid residue 62 of SEQ ID NO: 4,
and, wherein the last residue of said fragment ends at a position selected from the group consisting of:
l) amino acid residue 247 of SEQ ID NO: 4;
m) amino acid residue 248 of SEQ ID NO: 4;
n) amino acid residue 249 of SEQ ID NO: 4;
o) amino acid residue 250 of SEQ ID NO: 4;
p) amino acid residue 251 of SEQ ID NO: 4;
q) amino acid residue 252 of SEQ ID NO: 4; and,
r) amino acid residue 253 of SEQ ID NO: 4.

18. The single chain IL-12 polypeptide of claim 1, wherein p40C comprises a polypeptide sequence at least 80% identical, at least 85% identical, at least 90% identical, at least 95% identical, at least 97% identical, at least 98% identical, at least 99% identical, or 100% identical to amino acids 289 to 322, amino acids 289 to 328, amino acids 299 to 322, or amino acids 299 to 328 of SEQ ID NO: 2.

19. (canceled)

20. The single chain IL-12 polypeptide of claim 1, wherein p40C comprises a fragment of the polypeptide sequence of SEQ ID NO: 2, wherein the first residue of said p40C fragment begins at a position selected from the group consisting of:
a) amino acid residue 289 of SEQ ID NO: 2;
b) amino acid residue 290 of SEQ ID NO: 2;
c) amino acid residue 291 of SEQ ID NO: 2;
d) amino acid residue 292 of SEQ ID NO: 2;
e) amino acid residue 293 of SEQ ID NO: 2;
f) amino acid residue 294 of SEQ ID NO: 2;
g) amino acid residue 295 of SEQ ID NO: 2;
h) amino acid residue 296 of SEQ ID NO: 2;
i) amino acid residue 297 of SEQ ID NO: 2;
j) amino acid residue 298 of SEQ ID NO: 2; and,
k) amino acid residue 299 of SEQ ID NO: 2,
and, wherein the last residue of said fragment ends at a position selected from the group consisting of:
l) amino acid residue 322 of SEQ ID NO: 2;
m) amino acid residue 323 of SEQ ID NO: 2;
n) amino acid residue 324 of SEQ ID NO: 2;
o) amino acid residue 325 of SEQ ID NO: 2;
p) amino acid residue 326 of SEQ ID NO: 2;
q) amino acid residue 327 of SEQ ID NO: 2; and,
r) amino acid residue 328 of SEQ ID NO: 2.

21. A polynucleotide comprising a nucleic acid sequence encoding the single chain IL-12 polypeptide of claim 1.

22. The polynucleotide of claim 21, wherein the polynucleotide comprises a nucleic acid sequence at least 80% identical, at least 80% identical, at least 85% identical, at least 90% identical, at least 95% identical, at least 97% identical, at least 98% identical, at least 99% identical, or 100% identical to nucleotides 67 to 1599 or nucleotides 1 to 1599 of SEQ ID NO: 9.

23. (canceled)

24. A vector comprising the polynucleotide of claim 21.

25. The vector of claim 24, wherein the vector further comprises a gene switch capable of regulating expression of the single-chain IL-12 polypeptide.

26. The vector of claim 25, wherein the gene switch is an EcR-based gene switch.

27. (canceled)

28. An isolated host cell or a non-human organism transformed or transfected with the vector of claim 24.

29. (canceled)

30. (canceled)

31. (canceled)

32. (canceled)

33. (canceled)

34. A method of treating a patient comprising administering an effective amount of the single chain IL-12 polypeptide of claim 1.

35. A method of treating a patient comprising administering an effective amount of the polynucleotide of claim 21.

36. (canceled)

37. (canceled)

38. A pharmaceutically acceptable composition comprising the polypeptide of claim 1.

39. (canceled)

40. (canceled)

41. (canceled)

42. A pharmaceutically acceptable composition comprising the polynucleotide of claim 21.
